(19) United States
(12) Patent Application Publication
(10) Pub. No.: US 2026/0184800 A1
(43) Pub. Date: Jul. 2, 2026

(54) Methods of Improving Recovery after Lung Resection

(71) Applicants: University of Maryland, Baltimore, Baltimore, MD (US); Mayo Foundation for Medical Education and Research, Rochester, MN (US)

(72) Inventors: Alexander Sasha Krupnik, Owings Mills, MD (US); Zongcheng Mei, Baltimore, MD (US); Elizabeth Jacobsen, Phoenix, AZ (US)

(73) Assignees: University of Maryland, Baltimore, Baltimore, MD (US); Mayo Foundation for Medical Education and Research, Rochester, MN (US)

(21) Appl. No.: 19/547,045

(22) Filed: Feb. 23, 2026

Related U.S. Application Data
(63) Continuation-in-part of application No. 18/655,163, filed on May 3, 2024.
(60) Provisional application No. 63/463,977, filed on May 4, 2023.
(60) Provisional application No. 63/608,988, filed on Dec. 12, 2023.

(51) Int. Cl. C07K 16/28; A61K 39/00; A61P 7/00

(52) U.S. Cl. CPC C07K 16/2866; A61P 7/00; A61K 2039/505; C07K 2317/24; C07K 2317/732; C07K 2317/76

(57) ABSTRACT

Provided herein are methods for inhibiting or preventing activation of eosinophil production in a subject after a pulmonary surgical or medical procedure. Monoclonal antibodies, for example, an anti-IL-5rα antagonistic antibody, such as benralizumab, lirentelimab, or dexpramipexole are administered to the subject to directly target the eosinophils to reduce the number resident in the lung after the procedure.

### CROSS-REFERENCE TO RELATED APPLICATIONS

This application is a continuation-in-part under 35 U.S.C. § 120 of pending non-provisional patent application U.S. Ser. No. 18/655,163, filed May 3, 2024, which claims benefit of priority under 35 U.S.C. § 119(e) of provisional application U.S. Ser. No. 63/608,988, filed Dec. 12, 2023, and of U.S. Ser. No. 63/463,977, filed May 4, 2023, the entireties of both of which are hereby incorporated by reference.

### FEDERAL FUNDING LEGEND

This invention was made with government support under grant numbers A1116501, A1145108, and HL166402 of the National Institutes of Health. The government has certain rights in the invention.

### BACKGROUND OF THE INVENTION

### Field of the Invention

The present invention relates to the fields of pulmonary medicine and surgical procedures. More particularly, the present invention relates to methods for inhibiting eosinophilia after a pulmonary surgical procedure.

### Description of the Related Art

The increasing age of the global population has led to a substantial rise in the number of pulmonary resections performed worldwide (1, 2), Such operations include the resection of lung cancer, removal of malignant tumors metastatic to the lung, as well as interventions for pulmonary infections or emphysema. Despite this increase in pulmonary resections, it is surprising how little is known about the systemic stress response after thoracic surgery and how limited the interventions are to improve postoperative recovery.

Pulmonary complications occur in as many as 50% of patients undergoing lung resections (3) and a large portion of patients have prolonged dyspnea that requires supplemental oxygen post-operatively, even if they were not receiving oxygen therapy before their operation (4, 5). In addition to such manageable forms of mild respiratory insufficiencies which prolong hospital stay and hinder recovery, severe respiratory failure post-lung resection can also occur. For example, post-pneumonectomy pulmonary syndrome is defined as severe and life-threatening respiratory distress that occurs 6 hours to 6 days after removal of a whole lung and is unrelated to cardiogenic factors (6). The incidence of this complication has been reported to be as high as 7% in patients undergoing right pneumonectomy, but similar complications can occur after operations where less pulmonary tissue is removed (7).

Despite its identification in 1942 (8), the etiology of post-pneumonectomy pulmonary syndrome is unknown. Once post-pneumonectomy pulmonary syndrome develops, however, mortality rates can exceed 50% (9). Some have suggested that this complication may develop due to excessive administration of intraoperative fluid, or pulmonary damage associated with excessive ventilatory volumes during surgery (9-12), while others dispute such notions (13). Observational studies have suggested that the course and outcome of post-pneumonectomy pulmonary syndrome may be improved by the administration of high dose corticosteroids (14), but a mechanism underlying the therapeutic benefit of such steroid treatment remains unknown.

The prior art is deficient in methods of improving recovery from pulmonary surgery. Particularly, the prior art is deficient in methods of inhibiting activation of lung-resident eosinophils after a lung resection. The present invention fulfills this long-standing need and desire in the art.

### SUMMARY OF THE INVENTION

The present invention is directed to a method for depleting an eosinophil population in a subject in need thereof. In the method an amount of an antibody directed against eosinophils in the population is administered at least one time to the subject.

The present invention also is directed to a method for inhibiting an onset of eosinophilia in a subject after a pulmonary medical procedure. In the method eosinophils are depleted from a population thereof in the subject after the pulmonary medical procedure.

The present invention is directed further to an antibody-mediated method for preventing activation of lung-resident eosinophils in a subject after a lung resection surgery. In the method an amount of an anti-IL-5rα antagonistic antibody effective to directly target the lung-resident esoinophils is administered to the subject.

The present invention is directed further still to a post-surgical method for treating lung tissue after a procedure involving one or both lungs in a subject. In the method an amount of a monoclonal antibody is administered to the subject effective to directly reduce the number of eosinophils in the lung tissue and concomitantly to prevent disinhibition of cytotoxicity of immature neutrophils therein.

Other and further aspects, features, benefits, and advantages of the present invention will be apparent from the following description of the presently preferred embodiments of the invention given for the purpose of disclosure.

### BRIEF DESCRIPTION OF THE DRAWINGS

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-11 show that lung resection stimulates systemic eosinophilia. FIGS. 1A-1D show eosinophils expansion expressed as % of all CD45+hematopoietic cells in the blood, right lung, and spleen after lung transplantation (FIG. 1A), lung resection (FIG. 1B), sham thoracotomy (FIG. 1C), or pancreatectomy (FIG. 1D) where n=3 to 8 mice/time point of blood, n=4 to 9 mice/time point of right lung, n=2 to 7 mice/time point of spleen. FIG. 1E is a graphic representation of total CD45+leucocytes, expressed as number per μl of blood, after left lung transplantation, left pneumonectomy or sham thoracotomy where n=3 to 5 mice/group/time point. FIGS. 1F-1G show intravascular vs tissue parenchyma resident eosinophils (defined as SiglecF^{hi}CD11 b⁺ leucocytes) Intraparenchymal eosinophils area is boxed and labeled. Such data was obtained by injecting Phycoerythrin (PE) labeled anti-CD45 antibody intravascularly into a mouse 7 days post left pneumonectomy (FIG. 1F) or a resting control (FIG. 1G), removing the lung 10 minutes after injection and staining with a combination of antibodies specific for SiglecF, CD11 b and CD45.2 conjugated to Allophycocyanin (APC). Thus, eosinophils labeled with both PE and APC are located within the vasculature while eosinophils stained with CD45.2 (APC) but not PE are inside the tissue parenchyma (labeled box). This is representative of 2 separate experiments. FIGS. 1H-11 show human peripheral blood eosinophils (defined as CD45⁺Siglec8⁺CD16⁻) expressed as % of all CD45+peripheral blood leucocytes after resection of the lung (FIG. 1H) or abdominal organs (FIG. 11). Lung resection patients were subject to either wedge or segmental/lobal resection while the abdominal operations ranged from pancreatic to colon or liver resections as indicated in the figure. Statistical analysis by 2-tailed Student's t test (FIGS. 1A-1D) and Kruskal-Wallis test (FIGS. 1H-11). ns, p>0.05, *P<0.05, **P<0.01, ***P<0.001.

FIGS. 2A-2H show stress induced eosinophil activation after lung resection. FIGS. 2A-2B show phenotypic changes in right lung-resident eosinophils in lung (FIG. 2A) and blood (FIG. 2B) on day 1 and 4 post left pneumonectomy as defined by surface expression of CD107a (Lamp-1), and CD69 in addition to intracellular iNOS. Data representative of three separate experiments. FIG. 2C shows phenotypic changes of human peripheral blood eosinophils on day 0 (prior to surgery) as compared to either day 1,3,4 or 6 post lung resections. Data representative of at least two patients stained for such markers of activation. FIGS. 2D-2G are graphic representation of bone marrow-resident eosinophil development (FIG. 2D) with gating strategy (FIGS. 2E-2F) and quantification of various eosinophil progenitors (FIG. 2G) expressed either as % of bone marrow or % Ki67+ or defined as IL5Ra+SiglecF+CCR3+ in resting compared to day 4 post left pneumonectomy (PNX) (FIG. 2H). n=5 to 19 per group. The statistically significant increase in mature eosinophils is outlined by a box in bold. Statistical analysis by 2-tailed Student's t test (FIGS. 2D-2H).

FIGS. 3A-3G show that eosinophil depletion improves pulmonary function after lung resection. FIG. 3A shows that diphtheria toxin treatment of PHIL mice results in a near complete depletion of eosinophils compared to littermate controls. Eosinophils defined as Siglec-F^{hi}CD11 b⁺. Data representative of three independent experiments. FIG. 3B shows survival and FIG. 3C shows stress (defined by weight loss and recovery) after right pneumonectomy in eosinophil sufficient or deficient mice. FIG. 3D shows that neutralization/antagonism of either CCR3 or Siglec-F (murine homolog of human Siglec-8) improved survival post right pneumonectomy compared to IgG-treated mice. n=5-20 per group. FIG. 3E shows oxygenation, defined as partial pressure of arterial O₂, and FIG. 3F shows pulmonary edema (defined as wet to dry weight ratio) in eosinophil depleted or eosinophil sufficient mice that had undergone right pneumonectomy six hours earlier. Resting unmanipulated C57BL/6 mice included in each experiment as control. FIG. 3G shows edema of other, non-lung, organs after right pneumonectomy in eosinophil depleted, sufficient or resting unmanipulated C57BL/6 mice. n=4-6 per group. Log-rank test (FIGS. 3B-3D) and 2-tailed Student's t test (FIGS. 3E-3G) used for statistical analysis. ns, p>0.05, *P<0.05, **P<0.01, ***P<0.001

FIGS. 4A-4S show that IL-7 mediates stress induced eosinophil maturation and contributes to deleterious outcomes after lung resection. FIG. 4A shows bone marrow IL-7 levels in either resting mice (day 0) or on day 1 and day 4 post left pneumonectomy (PNX) and FIGS. 4B-41 show bone marrow levels of cytokines IL-5, IL-6, IFN-γ, TNF-α, IL-4, IL-18, IL-9, and IL-33, respectively. n=5 per group. FIG. 4J shows bone marrow-resident eosinophil development expressed by quantification of eosinophil progenitors or mature eosinophils expressed as % of bone marrow in either anti-IL7 mAb treated (IL-7 NT) or IgG control (ctrl) in resting or day 4 post left PNX mice. n=4-6 per group. FIG. 4K shows bone marrow-resident eosinophil development expressed by quantification of eosinophil progenitors or mature eosinophils expressed as % of bone marrow in either resting or day 4 post left PNX in IL-7 receptor deficient (IL-7R^{-/-}) or wild-type mice. n=4-6 per group. FIG. 4L shows IL-7 levels in the lung of either resting mice or day 1 and day 4 post left pneumonectomy (PNX). n=5 per group. FIG. 4M shows phenotypic changes in right lung-resident eosinophils on day 1 post left pneumonectomy as defined by surface expression of CD107a (Lamp-1), CCR3 and intracellular iNOS in either IL7R^{-/-} or wild-type mice. Data representative of three separate experiments. FIG. 4N shows survival post right pneumonectomy (PNX) of wild-type, IL7R^{-/-}, or wild-type mice in the presence of IL-7 neutralization. n=5-10 per group. FIG. 4O shows quantification of IL-7 production in the right lung, bone marrow, mediastinal lymph nodes and kidney using an IL-7^{GFP} reporter mouse. Based on the dim signal of endogenous GFP the intensity in the fluorescein (FITC) channel was amplified utilizing an anti-GFP antibody conjugated to FITC. Thus, background staining is plotted as anti-GFP staining in wild-type C57BL/6 mouse (grey) while black line denotes staining in resting IL-7^{GFP} mouse and red line is staining in IL-7^{GFP} mouse day 1 post left pneumonectomy. Data representative of three separate experiments. FIG. 4P shows quantification of IL-7 production of different subtypes of cells in the right lung of resting or day 1 post left PNX IL-7^{GFP} reporter mouse. Data representative of three separate experiments. FIG. 4Q shows quantification changes of IL-7 production derived from γ/δ T cells in the right lung of resting IL-7^{GFP} mouse (black), day 1 post left PNX IL-7^{GFP} mouse (red), pre-treated anti-TCR γ/δ cells mAb (γ/δ T depletion) IL-7^{GFP} mouse (blue), and day 1 post left PNX with prior treated anti-TCR γ/δ cells mAb (γ/δ T depletion) IL-7^{GFP} mouse (purple). Data representative of three separate experiments. FIG. 4R shows bone marrow and FIG. 4S shows lung eosinophil development expressed by quantification of mature eosinophils as % of hematopoietic cells in either anti-TCR γ/δ cells mAb treated (γ/δ T depletion) or IgG control (ctrl) treated mice at rest or day 4 post left PNX. n=4-6 per group. 2-tailed Student's t test and log-rank test. ns=p>0.05; *=p<0.05; **=p<0.01; ***=p<0.001.

FIGS. 5A-5F show IL-7 mediated eosinophil activation relies on ILC2s. FIG. 5A shows depletion of B cells or T cells does not affect stress induced eosinophil activation in the right lung after left pneumonectomy (PNX), as defined by upregulation of iNOS or CD107a. Data representative of three separate experiments. FIG. 5B shows quantification of eosinophils in the right lung in resting or day 1 or 4 post left pneumonectomy (PNX) in mice depleted of B cells (pre-treated with anti-CD20 mAb (purple)), or depleted of T cells (pre-treated with anti-CD4 plus anti-CD8 mAb (orange)) vs. control mice treated with IgG control (black). n=3-6 per group. FIG. 5C shows quantification of eosinophils in the bone marrow in resting or 4 days post left pneumonectomy (PNX) mice, pre-treated with either anti-CD20 mAb (purple), anti-CD4 plus anti-CD8 mAb (orange) or IgG control (black). n=5 per group. FIG. 5D shows depletion of ILC2s, using a tamoxifen induced Cre-mediated deletion of the transcription factor Gata-3, ameliorated stress induced eosinophil activation as measured by iNOS and CD107a expression. Data representative of three separate experiments. FIG. 5E shows quantification of eosinophils in the right lung in resting or 1 vs. 4 days post left pneumonectomy (PNX), in mice pre-treated with either tamoxifen (cyan) or saline control (black). n=3-6 per group. FIG. 5F shows quantification of eosinophils in the bone marrow in resting or 4 days post left pneumonectomy (PNX) mice, pre-treated with either tamoxifen (cyan) or saline control (black). n=5 per group. 2-tailed Student's t test. ns=p>0.05; *=p<0.05; **=p<0.01; ***=p<0.001.

FIGS. 6A-6F shows that ILC2s are activated after lung resection. FIG. 6A shows qualitative and quantitative analysis of ILC2 in the right lung and bone marrow of wild-type mice day 1 and day 4 post left pneumonectomy (PNX). Data representative of three separate experiments. FIG. 6B shows ILC2 activation, as measured by PD-1, KLRG-1, and GM-CSF levels on day 1 post left pneumonectomy (PNX) in the presence or absence of IL-7 neutralization. Unlike previous methodology, where IL-7 was neutralized at least four days before resection resulting in the death of ILC2, for this set of experiments a one-time dose of IL-7 neutralizing antibody was given at the time of resection with analysis on post-operative day one. Data representative of three separate experiments. FIG. 6C shows quantification of eosinophils in the bone marrow in resting or 4 days post left pneumonectomy (PNX) mice, post adoptive transfer of wild-type ILC2s (purple) or saline control (black). n=4-6 per group. FIG. 6D shows quantification of eosinophils in the bone marrow in resting or day 4 post left pneumonectomy (PNX) mice, post adoptive transfer with GM-CSF^{-/-} ILC2s (orange) or saline control (black). n=4-6 per group. FIG. 6E shows phenotypic changes of eosinophils activation, as defined by upregulation of iNOS or CD107a, in the right lung in resting or day 1 post left pneumonectomy (PNX) mice, post adoptive transfer of wild-type ILC2s, GM-CSF^{-/-} ILC2s or saline control. Data representative of three separate experiments. FIG. 6F shows survival of GM-CSF^{-/-as compared to wild-type mice after right pneumonectomy (PNX). n=}10-15 per group. 2-tailed Student's t test (FIGS. 6C-6D) and log-rank test (FIG. 6F). ns=p>0.05; *=p<0.05; **=p<0.01; ***=p<0.001

FIGS. 7A-7E show that eosinophil-associated iNOS contributes to pulmonary pathology after lung resection. FIG. 7A shows that after lung resection eosinophils express the highest levels of iNOS compared to other cell types and FIG. 7B shows that eosinophil depletion, utilizing diphtheria administration in iPHIL mice, eliminated the highest iNOS-expressing population. Data representative of three separate experiments. FIG. 7C shows nitrotyrosine immunohistochemistry in the left lung of resting C57BL/6 mice or 6 hours after right pneumonectomy (PNX). Data representative of three separate experiments. Scale bars, 100 μm. FIG. 7D shows survival of iNOS^{-/-} as compared to wild-type mice after right pneumonectomy (PNX). FIG. 7E shows survival of mice pre-treated with one-time dose of the NOS inhibitor L-NAME three hours prior as compared to wild-type mice after right pneumonectomy (PNX). log-rank test (FIG. 7D-7E). n=10 n=10-15 per group. per group. ns=p>0.05; *=p<0.05; **=p<0.01; ***=p<0.001

FIGS. 8A-8C show depletion of IL-5 Receptor alpha (IL-5Ra) Expressing Cells Improves Survival while IL-5 Neutralization Decreases Survival after Pulmonary Resection. FIG. 8A shows mouse survival after right pneumonectomy and treatment with control IgG (black round circles), IL-5Ra depleting clone H7, murine analog of FASENRA (benralizumab) (purple circle) or IL-5 neutralizing clone TRFK5, murine analog of NUCALA (mepolizumab) or CINQAIR (reslizumab). FIG. 8B shows the validation of eosinophil depletion after treatment with control IgG, TRFK5 or H7. Eosinophils identified as SiglecF^{hi}CD11b^{hi} cells. FIG. 8C shows the protein concentration in the bronchioalveolar lavage (BAL) 4 hours after right pneumonectomy and treatment with either TRFK5 or H7. *=p<0.05, **=p<0.01.

FIGS. 9A-9F show that pulmonary resection leads to a rapid influx of IL-5 Receptor alpha (IL-5Rα) expressing neutrophils into the lung. FIG. 9A shows the percentage of IL-5Rα expressing cells expressed as % of all live cells in various murine tissues. FIG. 9B shows relative percentage of cell types expressing IL-5Rα in various murine tissues. FIG. 9C shows the changes in the relative composition of hematopoietic cells in the left lung four hours after right lung resection. FIG. 9D shows the neutrophil penetration into the pulmonary parenchyma as defined by staining with a fluorochrome conjugated anti-CD45 antibody administered i.v. This is followed by tissue harvest and staining with neutrophil specific markers as well as anti-CD45.2 conjugated to a different fluorochrome. CD45.2^{hi}CD45^{low} neutrophils are defined as located within the parenchyma and not in direct contract with blood. FIG. 9E shows the quantification of neutrophils, defined as % of all live cells, in the blood and bone marrow four hours after right pneumonectomy (RPX). FIG. 9F shows the quantification of neutrophils, defined as % of all live cells, in the left lung four hours after right pneumonectomy (RPX).

FIGS. 10A-10E show that IL-5Rα expressing neutrophils depend on G-CSF for activation. FIG. 10A shows the survival of mice subject to right pneumonectomy with or without neutrophil depletion in the presence (top) or absence of the cytokine IL-5 (bottom). FIG. 10B shows the cytokine levels in the lung after IL-5 neutralization, Right PNX, or both. FIG. 10C shows the survival of mice subject to right pneumonectomy with or without neutralization or depletion of select cytokines and chemokines in the presence (top) or absence of the cytokine IL-5 (bottom). FIG. 10D shows the quantification of IL-5Rα−(top) or IL-5Rα+(bottom) neutrophils, defined as % of all live cells, in the left lung four hours after right pneumonectomy (RPX), IL-5 neutralization and neutralization of either CXCR2 (light blue) or G-CSF (purple). FIG. 10E shows the phenotype of IL-5Rα−(top) or IL-5Rα+(bottom) neutrophils with or without G-CSF neutralization. ns=p>0.05, *=p<0.05, **=p<0.01, ***=p<0,001.

FIGS. 11A-11E shows that IL-5Rα expression identifies an immature population of neutrophils that express markers of activation and cytotoxicity at baseline. FIG. 11A shows the percent of organ resident neutrophils expressing the IL-5Rα receptor at baseline. FIG. 11B shows the expression of activation and cytotoxicity markers of IL-5Rα−(black) or IL-5Rα+(red) neutrophils in the lung (top) or bone marrow (bottom). FIG. 11C shows the activation markers on IL-5Rα−(black) or IL-5Rα+(red) neutrophils blood of a healthy volunteer. FIG. 11D shows the quantity (left) and activation status of IL-5Rα+expressing neutrophils in the blood before (black) and four hours after (red) lung resection in man. FIG. 11E shows the co-expression of CD101 and IL-5Rα on neutrophils.

FIGS. 12A-12E show that IL-5 inhibits activation of IL-5Rα expressing neutrophils through multiple overlapping pathways. FIG. 12A shows the phosphorylation of Jak2, Stat3 (as determined by phospho-flo staining) and IL-1p levels in IL-5Rα+neutrophils cultured under various conditions of G-CSF and IL-5.

FIG. 12B shows the activation of IL-5Rα+neutrophils (as measured by IL-1p expression) in the presence of the Jak2 inhibitor Fedratinib or FIG. 12C when activated by LPS rather than G-CSF. FIG. 12D shows high resolution confocal microscopy for G-CSF receptor (green), IL-5Rα (orange) and phosphoJak2 (purple). FIG. 12E shows the SOCS3 and G-CSFR expression in IL-5Rα+ or IL-5Rα−neutrophils after exposure to IL-5 or not.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

As used herein, the conditional language, such as, among others, “can”, “might”, “may”, “e.g.”, “for example”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

As used herein, the term “subject” refers to any person having had a pulmonary medical or surgical procedure who is the recipient of the treatments provided herein.

As used herein, the terms “therapeutic agent”, inhibitory agent” “eosinophil targeting agent” and “targeting agent” are used interchangeably.

In one embodiment of the present invention, there is provided a method for method for depleting an eosinophil population in a subject in need thereof, comprising administering at least one time to the subject an amount of an antibody directed against eosinophils in the population.

In this embodiment, the antibody may directly target and kill the eosinophil population. Also in this embodiment, the antibody may be a monoclonal antibody. In one aspect, the monoclonal antibody may be an anti-IL-5Rα antagonistic antibody. In another aspect, the monoclonal antibody may be selected from the group consisting of benralizumab, lirentelimab, and dexpramipexole. In addition, the subject in need underwent a medical procedure involving one or both lungs. Particularly, the medical procedure may be a lung resection, a lung transplantation, radiofrequency ablation of pulmonary tumors or radiotherapy of pulmonary tumors.

In another embodiment of the present invention, there is provided a method for inhibiting an onset of eosinophilia in a subject after a pulmonary medical procedure, comprising depleting eosinophils from a population thereof in the subject after the pulmonary medical procedure.

In this embodiment, the depleting step may comprise targeting directly the eosinophils in the population with an antibody effective to reduce the number of the eosinophils therein. Also in this embodiment, the antibody may target a cytokine associated with the eosinophil activation cascade. In addition, the antibody may be an anti-IL-5Rα antagonistic antibody. Particularly, the anti-IL-5Rα antagonistic antibody may be selected from the group consisting of benralizumab, lirentelimab, and dexpramipexole. Furthermore, the pulmonary medical procedure is a lung resection.

In yet another embodiment of the present invention, there is provided an antibody-mediated method for preventing activation of lung-resident eosinophils in a subject after a lung resection surgery, comprising administering to the subject an amount of an anti-IL-5rα antagonistic antibody effective to directly target the lung-resident esoinophils.

In one aspect of this embodiment, the anti-IL-5rα antagonistic antibody may be a monoclonal antibody. In another aspect, the antibody may be selected from the group consisting of benralizumab, lirentelimab, and dexpramipexole.

In yet another embodiment of the present invention, there is provided a post-surgical method for treating lung tissue after a procedure involving one or both lungs in a subject, administering an amount of a monoclonal antibody to the subject effective to directly reduce the number of eosinophils in the lung tissue and concomitantly to prevent disinhibition of cytotoxicity of immature neutrophils therein. In this embodiment, the monoclonal antibody may be an anti-IL-5rα antagonistic antibody that directly targets the eosinophils; where the antibody is not an anti-IL-5 antagonistic antibody. Particularly, the monoclonal antibody may be benralizumab. Also in this embodiment, the procedure may be a lung resection, a lung transplantation, radiofrequency ablation of pulmonary tumors or radiotherapy of pulmonary tumors.

In yet another embodiment of the present invention, there is provided a method for preventing activation of eosinophils in a subject in need thereof, comprising administering at least one time to the subject an amount of a therapeutic agent effective to inhibit eosinophil activation.

In this embodiment, the therapeutic agent may inhibit activation of lung-resident eosinophils or inhibits delayed stress-induced eosinophil maturation in bone marrow. In this embodiment, the subject in need underwent a medical procedure involving one or both lungs. Particularly, the medical procedure may be a lung resection, a lung transplantation, radiofrequency ablation of pulmonary tumors or radiotherapy of pulmonary tumors.

In this embodiment, the therapeutic agent may be an antibody, a chemical compound, or a protein, or a combination thereof. In one aspect of this embodiment, the antibody may be a monoclonal antibody selected from the group consisting of Mepolizumab, Resilzumab, Benralizumab, Antolimab, Depemokimab, Tezepelumab, Dupilumab, Tralokinumab, Beritilimumab, Itepekimab, Astegolimab, Tozorakimab, Melrilimab, Lebrikizumab, Romilkimab, and Cendakimab or the antibody may be an engineered antibody selected from the group consisting of PF-07275315, PF-0726264660, and SAR443765. In another aspect of this embodiment, the chemical compound may be dexpramipexole dihydrochloride or is a nitric oxide synthase inhibitor N(gamma)-nitro-L-arginine methyl ester. In yet another aspect, the protein may be an anticalin or may be a fusion protein bizaxofusp.

In yet another embodiment of the present invention, there is provided a method for inhibiting an onset of eosinophilia in a subject after a pulmonary medical procedure, comprising administering an effective amount of an eosinophil targeting agent to the subject.

In this embodiment, the pulmonary medical procedure may be a lung resection, a lung transplantation, radiofrequency ablation of pulmonary tumors or radiotherapy of pulmonary tumors. Also, in this embodiment, the eosinophil targeting agent may be effective to inhibit an eosinophil activation cascade.

In an aspect of this embodiment, the eosinophil targeting agent targets at least one cytokine associated with the eosinophil activation cascade selected from the group consisting of IL-5, IL-5Rα, IL-4, IL-5Rα, IL-13, IL-33, C—C motif chemokine 11 (CCL11), and thymic stromal lymphopoietin (TSLP). Representative examples of the eosinophil targeting agent are selected from the group consisting of Mepolizumab, Resilzumab, Benralizumab, Depemokimab, Tezepelumab, Dupilumab, Tralokinumab, Beritilimumab, Itepekimab, Astegolimab, Tozorakimab, Melrilimab, Lebrikizumab, Romilkimab, Cendakimab, PF-07275315, PF-0726264660 and SAR443765. In another aspect, the eosinophil targeting agent may inhibit production of eosinophil-derived nitrous oxide (NO). A representative example of the eosinophil targeting agent is N(gamma)-nitro-L-arginine methyl ester. In yet another aspect, the eosinophil targeting agent may inhibit at least one of a chemokine receptor-3 (CCR3), an ST2 receptor or an Siglec-8 inhibitory receptor associated with the eosinophils. Representative examples of the eosinophil targeting agent may be at least one of the monoclonal antibody Beritilimumab, Astegolimab or Antolimab.

In yet another embodiment of the present invention, there is provided a method for preventing activation of lung-resident eosinophils in a subject after a lung resection surgery, comprising administering to the subject an amount of an inhibitory agent effective to target at least one step of an eosinophil activation pathway.

In an aspect of this embodiment, the inhibitory agent may be a monoclonal antibody selected from the group consisting of Mepolizumab, Resilzumab, Benralizumab, Antolimab, Depemokimab, Tezepelumab, Dupilumab, Tralokinumab, Beritilimumab, Itepekimab, Astegolimab, Tozorakimab, Melrilimab, Lebrikizumab, Romilkimab, and Cendakimab or is an engineered antibody selected from the group consisting of PF-07275315, PF-0726264660 and SAR443765. In another aspect of this embodiment, the inhibitory agent may be a chemical compound N(gamma)-nitro-L-arginine methyl ester or dexpramipexole dihydrochloride or a protein that is anticalin or bizaxofusp.

The present invention describes that eosinophils mediate respiratory insufficiency after pulmonary resection. Lung resection, but not surgical removal of other organs, triggers a process that's defined as “stress-induced maturation and activation” of eosinophils. Surgical stress results in increased activation of mature eosinophils and accelerates the rate of eosinophil production in the bone marrow. The present invention demonstrates that IL-7-driven activation of ILC2s, and their subsequent production of GM-CSF, are critical mediators of eosinophilic activation. Activated eosinophils induce pulmonary damage through production of iNOS and nitrosylation of residual lung tissue. Disruption of this process at any of its multiple steps can improve both respiratory function and survival following pulmonary resection.

Thus, provided herein are methods for preventing or inhibiting activation of eosinophils, particularly lung-resident eosinophils, in a subject after a pulmonary surgery or other pulmonary medical procedure. For example, the pulmonary surgical or medical procedure may be, but is not limited to, a lung resection, a lung transplantation, radiofrequency ablation of pulmonary tumors or radiotherapy of pulmonary tumors.

Inhibition is achieved by blocking, antagonizing, targeting or by other inhibitory means one or more steps in the eosinophil activation pathway. For example, a therapeutic agent, an inhibitory agent or targeting agent may be administered to the post-surgical subject that is effective to interfere with, for example, but not limited to, one or more steps in the IL-7/ILC2/GM-CSF axis in an eosinophil activation pathway. Representative therapeutic, inhibitory or targeting agents or compounds may inhibit various cytokines, for example, but not limited to, interleukins IL-5, IL-5Rα, thymic stromal lymphopoietin (TSLP), IL-4R, IL-4Ra, IL-13, or IL-33, may inhibit the chemokines may target or neutralize production of eosinophil-derived nitrous oxide, such as with N(gamma)-nitro-L-arginine methyl ester (L-NAME) or may target eosinophil production, such as by inhibiting the chemokine C—C motif chemokine 11 (CCL11) and/or its chemokine receptor-3 (CCR3) and/or inhibiting the IL-33 receptor ST2 and/or inhibiting the Siglec-8 inhibitory receptor found on eosinophils. Representative eosinophil depleting agents are shown in Table 1.

TABLE 1

| | | |
|---|---|---|
| Name | Molecular Target | Molecule Type |

Directly depletes eosinophils and reduce migration/activation state

| | | |
|---|---|---|
| Mepolizumab | IL-5 | Monoclonal antibody |
| Resilzumab | IL-5 | Monoclonal antibody |
| Benralizumab | IL-5Rα | Monoclonal antibody |
| Antolimab | Siglec-8 | Monoclonal antibody |
| Dexpramipexole | NA | chemical |
| dihydrochloride | | |
| Depemokimab | IL-5 | Monoclonal antibody |

Indirectly reduce eosinophil migration/activation/survival state

| | | |
|---|---|---|
| Tezepelumab | TSLP | Monoclonal antibody |
| Dupilumab | IL-4R□ | Monoclonal antibody |
| Tralokinumab | IL-13 | Monoclonal antibody |
| Beritilimumab | CCL11 | Monoclonal antibody |
| Itepekimab | IL-33 | Monoclonal antibody |
| Astegolimab | ST2 | Monoclonal antibody |
| Tozorakimab | IL-33 | Monoclonal antibody |
| Melrilimab | IL-33 | Monoclonal antibody |
| Anticalin | IL-4R□ | Protein |
| Lebrikizumab | IL-13 | Monoclonal antibody |
| Romilkimab | IL-4, IL-13 | Bispecific antibody |
| Bizaxofusp | IL-4R | Fusion protein with toxin |
| Cendakimab | IL-13 | Monoclonal antibody |
| PF-07275315 | IL-4/IL-13/TSLP | Engineered antibody |
| PF-0726264660 | IL-4/IL-13/IL-33 | Engineered antibody |
| SAR443765 | IL-13/TSLP | Antibody |
| N(gamma)-nitro-L- | Nitrous oxide | Chemical |
| arginine methyl | production | |
| ester | | |

These eosinophil targeting or inhibitory agents may be administered to the subject at least once on a dosing schedule determined by one of ordinary skill in the art. One of ordinary skill in pulmonary surgery or other pulmonary medical procedures is well able to determine the dose, dosing schedule based on the subject's age, sex, general health prior to surgery, the condition requiring surgery, and the post-surgical condition of the subject. The targeting agent and inhibitory agents may be administered as pharmaceutical compositions or immunogenic compositions with an appropriate carrier, such as, a pharmaceutically acceptable carrier, an adjuvant or excipient as well-known in the art. The targeting or inhibitory agents dosage is effective to produce a pharmacologic, immunologic or therapeutic result that prevents or decreases the production of eosinophils in the lungs of the subject post-surgery.

Also provided are methods to improve recovery after lung resection to those directly depleting eosinophils through targeted cytotoxicity rather than those that facilitate eosinophil death through depletion of the cytokine IL-5. An example of such a method is the antibody-mediated direct targeting of eosinophils, such as can occur through the administration of anti-IL-5Rα antagonistic antibodies. One such example is benralizumab (FASENRA). Other examples of direct targeting agents include agents targeting Siglec-8, for example, lirentelimab, dexpramipexole, and others.

The following examples are given for the purpose of illustrating various embodiments of the invention and are not meant to limit the present invention in any fashion.

### Example 1

### Materials and Methods

### Study Design

For in vivo experiments, a minimum of two independent repeats of experiments were performed. For in vitro experiments, a minimum of three biological repeats were performed. All data collected were included for quantification and analysis. All experiments were randomized and blinded when possible. Mice were grouped by similar age, sex, and weight. Sample sizes are indicated in figure legends. Histology graphs are analyzed by our group and results are confirmed by an experienced pathologist. All animal studies were approved by the Institutional Animal Use and Care Committee at University of Maryland, Baltimore and performed according to the guidelines. Human studies were performed under IRB #HP-00101225.

### Mice

Male Balb/c, C57BL/6 (B6), B6.129S7-II7r^{tm1mx/J}(IL-7Rα^{-/-}), B6.129S-Csf2^{tm1MIg/J}(GM-CSF^{-/-}), B6.129P2-Nos2^{tm1Lau/J}(iNOS^{-/-}) and B6.SJL/BoyJ CD45.1 congenic mice were purchased from the Jackson Laboratory (Bar Harbor, ME). C57BL/6 iPHIL (inducible eosinophil depletion strain (EPX-DTR) (21) were bred and maintained in a colony. IL-7-eGFP mice (C57BL/6 background) were provided by Dr. McCune and Dr. Corey of UCSF (99) were bred and maintained. GATA3creERT2 (ILC2^{-/-}) (C57BL/6 background), were provided by Dr. Jinfang Zhu (NIH/NIAID) were bred and maintained. All mice were kept in the same room of the same vivarium (Program in Comparative Medicine, University of Maryland, Baltimore) after delivery from vendors with the same diet and water supply before being used for each experiment.

### Surgery

Left pneumonectomies in mice were performed by opening the left-side chest cavity, ligating the left hilum and removing the entire left lung. Right pneumonectomies in mice were performed by opening the right-side chest cavity, ligating the right hilum and removing the entire right 4 lobes, including the mediastinal lobe. To prevent hemodynamic instability after a right pneumonectomy 300 ul of air was withdrawn from right-side chest cavity after closing the ribcage. Sham thoracotomies and pancreatic resections were performed on wild-type mice. Pancreatic resections were performed by removing pancreas left (tail) part. Orthotopic left lung transplants were performed using the Balb/c a C57BL/6 strain combination as described throughout the text according to previously defined protocols (100). Animals exhibiting severe distress or losing 20% of their body weight received frequent monitoring and, if symptoms did not resolve, were euthanized. Animals that were sacrificed due to these parameters were considered as succumbing to the operative treatment conditions.

### In Vivo Antibodies and Chemicals

Most of in vivo used antibodies were purchased from BioXcell and given intraperitoneally (i.p.). Transplant studies included treatment with co-stimulatory blockade (CSB) consisting of 250 μg of anti-CD40L Abs (MR1, Catalog #BE0017-1, BioXcell) on POD 0 and 200 μg of mouse recombinant CTLA4 Abs (Catalog #BE0099, BioXcell) on POD 2 as described previously (100). Depletion of eosinophils in iPHIL mice was accomplished as described (22). Diphtheria toxin was purchased from Sigma, Catalog #D0564-1MG. Depletion of ILC2 in GATA3creERT2 mice was accomplished by i.p. injection of tamoxifen, purchased from Sigma, Catalog #85256-50 mg. Tamoxifen was given 2 mg every other day, 3 doses in total, then rested for 2 more weeks. Depletion of T cells was accomplished by combination of anti-CD4 antibody (Catalog #BE0003-1, BioXcell, clone GK1.5, 200 ug/dose, 3 consecutive days prior to surgery) and anti-CD8 antibody (Catalog #BE0117, BioXcell, clone YTS169.4, 200 ug/dose, 3 consecutive days prior to surgery). Depletion of B cells was accomplished by CD20 antibody (Catalog #BE0356, BioXcell, clone MB20-11, 100 ug/dose, 3 consecutive days prior to surgery). Blockade of IL-5 was accomplished by IL-5 neutralization antibody (Catalog #BE0198, BioXcell, clone TRFK5, 200 ug/dose, 4 consecutive days, 2 doses pre-surgery and 2 doses post-surgery). Blockade of IL6R was accomplished by IL-6R blockade antibody (Catalog #BE0047, BioXcell, clone 15A7, 200 ug/dose, 4 consecutive days, 2 doses pre-surgery and 2 doses post-surgery). Blockade of IL-7 was accomplished by IL-7 neutralization antibody (Catalog #BE0048, BioXcell, clone M25, 600 ug/dose, first dose was given 4 days prior to surgery and followed by 2 days and 1 day prior to surgery, 1 day and 2 days post-surgery, for a total 5 doses). Blockade of IL-9 was accomplished by IL-9 neutralization antibody (Catalog #BE0181, BioXcell, clone 9C1, 100 ug/dose, 4 consecutive days, 2 doses pre-surgery and 2 doses post-surgery). CD45 Antibody (clone 30-F11) was diluted in phosphate buffered saline (PBS) and injected intravenously 5 mins before mice were euthanized. CCR3-blockade/depletion (clone 6S2-19-4, BioXcell) was accomplished as previously described by administering 200 ug/mouse 4 days prior to right pneumonectomy i.p. (101). SiglecF targeting/antagonism (clone #238047, R&D Systems) was accomplished by administrating 40 ug i.p. 24 hours prior to right pneumonectomy as previously described (102). N(gamma)-nitro-L-arginine methyl ester (L-NAME) was purchased from Millipore-Sigma (catalog #N5751) and administered i.p. at 50 ug/kg as a one-time dose 3 hours prior to resection. yb T cells depletion (clone UC7, BioXcell) was accomplished by administering 400 ug/mouse 2 days and 1 day prior to right pneumonectomy i.p.

### Arterial Blood Qas and Wet to Dry Weiqht Ratio Determination

Six hours after right pneumonectomy, arterial blood was drawn from the ascending aorta while mice were ventilated with room air. Blood gases were measured using an iSTAT Portable Clinical Analyzer (iMale STAT Corp, East Windsor, NJ). Mice organs were dissected and weighed before and after drying at 60° C. for 48 hours.

### Preparation of Bone Marrow and Lung Tissue

Bone marrow cell isolation was performed by cutting proximal and distal femur edges and flushing with PBS plus 10% FBS solution in a syringe. This plug was gently crushed and strained to get single cell suspension. Lung tissue was digested by collagenase and DNase as previously described (55). Briefly, lung tissue was well minced with scissors and digested by placing them into RPMI 1640 medium (Thermo Fisher) containing 0.5 mg/ml collagenase II (Worthington Biochemical Corporation) and 5 U/ml DNase (Millipore Sigma) for 35 minutes at 37° C. in a shaker. The digested lung tissue was passed through a 70-μm cell strainer and treated with ACK lysing buffer (Lonza) to remove red cell contamination.

### Adoptive Transfer

ILC2s were isolated from lungs of C57BL/6 wild type, B6.SJL/BoyJ CD45.1 congenic mice or GM-CSF−/−mice pre-treated with IL-33 (500 ng in 40 ul PBS by intratracheal delivery on days 0 and 2) and then harvested for single cell isolation on day 5. ILC2s were isolated and expanded as described previously (71). ILC2s (500,000 in total) were given by intravenous injection 1 day before surgery.

### Histology Analysis

For H&E staining, lung tissue was harvested and fixed for 2 days in 10% buffered formalin (Thermo Fisher Scientific) and then transferred to 70% ethanol. Samples were embedded in paraffin and then stained by H&E per established methods. For immunofluorescence experiments, mouse lungs were harvested and frozen in OCT (Sakura Finetek) on dry ice. Specifically, lung tissue was intratracheally and interstitially injected with a 10% formalin/OCT (1:1) solution before being frozen on OCT. 7 μm cryosections were fixed with cold acetone/methanol (1:1) solution for 5 minutes. Antibodies were diluted according to the manufacturer's protocol. After staining with primary antibodies, sections were blocked with 10% serum of the secondary antibody host and incubated with secondary antibodies for 60 minutes. Slides were fixed with 4% PFA solution followed by 1% glycerol incubation for 5 minutes, respectively. ProLong Gold Antifade Mountant (catalog P36930. Thermo Fisher Scientific) was added before putting the cover slides on. Images were acquired with the EVOS FL Auto 2 and Leica DM6 B Imaging system and analyzed with LAS X analysis software (Leica). Primary antibodies used consisted of rabbit anti-nitrotyrosine antibody (1:200, Catalog #A-21285; Thermo Fisher). Secondary Antibodies consisted of APC donkey anti-rabbit IgG (1:400, Jackson ImmunoResearch).

For immunohistochemistry, formalin-fixed, 5 μm sections of paraffin-embedded specimens were deparaffinized and rehydrated. Following antigen retrieval in citrate buffer (pH 6.0, Dako), endogenous peroxide activity was quenched with 3% H2O2. HRP-DAB TUNEL assay kit (Catalog #ab206386) was used for TUNEL staining.

### Flow Cytometry

Saturating concentrations of fluorochrome-conjugated antibodies were used in all flow cytometric analysis. Most antibodies were purchased from BD Biosciences (San Jose, CA), BioLegend (San Diego, CA) or eBioscience (ThermoFisher Scientific, San Diego, CA). Unless otherwise indicated all staining was performed by adding 1:100 dilution of the fluorochrome-conjugated antibody to 0.5-1×106 cells and stained at 4° C. for 30-45 min in 100 ul FACS buffer consisting of phosphate buffered saline with 5% fetal calf serum. Excess antibody was removed by two consecutive washings. All surface staining was performed on ice in staining buffer (2% FCS, 0.1% NaN3 in PBS) containing anti-FcR antibodies clone (2.4G2). Native or allograft lung tissue was minced, placed in RPMI 1640 medium (Thermo Fisher, MA) containing 0.5 mg/ml collagenase II (Worthington Biochemical Corporation, NJ) and 5U/ml DNase (Millipore Sigma, MA), and homogenized in a tissue dissociator (Miltenyi, MD). Tissue suspensions were incubated at 37° C. for 40 minutes. The digested lung tissue was passed through a 70 mm strainer and treated with ACK buffer. Cells were stained with the LIVE/DEAD Fixable Yellow Stain kit (Thermo Fisher, MA) followed by fluorochrome-labeled antibodies. For some experiments mice were injected with 500 ug brefeldin 6 hours before tissue harvest as previously described (103). Cells were subsequently stained with surface antibodies, followed by 25-minute fixation and permeabilization and intracellular antibodies staining. In experiments involving intranuclear markers, lung cells were permeabilized with the Foxp3/Transcription Factor Fixation/Permeabilization buffer (Thermo Fisher, MA) before adding intranuclear antibodies. Sample data was acquired on an Aurora (Cytek Biosciences, CA) and analyzed using FlowJo v10.

Most antibodies and their isotype controls were purchased from BD, Biolegend or Thermo Fisher Scientific. anti-mouse CD11b (clone M1/70), anti-mouse CD45.2(clone 104), anti-mouse CD45(clone 30-F11), anti-mouse CD107a (clone LAMP-1), anti-mouse iNOS (clone CXNFT), anti-mouse CD69 (clone H1.2f3), Lin cocktail includes anti-mouse CD3 (clone 145-2c11), anti-mouse CD19 (clone 1D3), anti-mouse CD4 (clone GK1.5), anti-mouse CD8 (clone 53-6.7), anti-mouse B220 (clone RA3-6B2), anti-mouse Gr1 (clone RB6-8C5), anti-mouse Ter119 (clone Ly-76), anti-mouse Sca-1 (clone D7). Anti-mouse CD34 (clone RAM34), anti-mouse IL-5Rα (clone T21), anti-mouse C-Kit (clone 2B8), anti-mouse CD16/32 (clone 93), anti-mouse NK1.1 (clone PK136), anti-mouse SiglecF (clone 1RNM44N), anti-mouse CCR3 (clone J073e5), anti-mouse CD127 (clone A7R34), anti-GFP(Invitrogen, polyclonal, Catalog #A-11122), anti-mouse CD90.2 (clone Thy-1.2), anti-mouse sca-1(D7), anti-mouse ST2 (clone RMST2-2), anti-mouse CD45.1 (clone A20), anti-mouse PD-1 (clone J43), anti-mouse klrg1 (clone 2F1), anti-mouse Gm-CSF (clone MP1-22E9), anti-mouse PDL-1 (clone M1H5), anti-mouse CD80 (clone 16-10A1), anti-mouse CTLA-4 (clone UC10-4B9), anti-mouse MHCII (clone m5/113.15.2), anti-mouse Tim3 (clone RMT3-23), anti-mouse CD11c (clone N418), anti-mouse γδ TCR (clone GL3).

### Human Eosinophil Evaluation

Human studies were performed under IRB #HP-00101225. Peripheral blood was collected at different intervals (right before surgery as well as at various time points ranging from one to six days post-surgery) and analyzed by flow cytometry. Antibodies used included: Anti-human CD45(clone H30), anti-human Siglec8(clone 7C9), anti-human CD16 (clone CB16), anti-human CD69(clone H1.2F3), anti-human CD107a (clone H4A3), anti-human CD19 (clone SJ25C1), anti-human CD63 (clone H5C6), anti-human CD3(clone UCHT1).

### Cytokine Detection

For some cytokines, such as IL-7, levels in lungs and bone marrow were measured using ELISA kits (R&D Systems, Minneapolis, MN, Catalog #DY407) per manufacturer instructions. For other cytokines multiplex analysis was used. Mouse lung tissue and bone marrow was lysed in RIPA buffer with phosphatase inhibitors and quantified with a Pierce BCA Protein Assay Kit (ThermoFisher, MA). Cell lysis buffer was used as matrix solution for the background, standard curve, and quality control samples. Sample data were acquired on a Millipore Sigma Magpix instrument and analyzed using the Milliplex Analyst software.

### In Vitro Eosinophil Differentiation

Eosinophils were differentiated from whole bone marrow as described previously (29, 79), where whole bone marrow is treated with FLT3 and SCF (100 ng/mi) for 4 days then washed and treated with IL-5 for 10 more days (10 ng/mL) with flasks changed on day 4, 8, 10, 12 to remove adherent cells. By Day 8, 40% of cells are eosinophils (Siglec-F+Cd11b⁺) and by day 10, >90% of the cells were eosinophils, and day 12-14 100% eosinophils. At Day 8 and Day 10, the population of immature eosinophils (CCR3 low) was 80% and 60%, respectively. By day 14 all cells are eosinophils, and all are CCR3 hi mature eosinophils (i.e., fully differentiated). IL-7 (20 ng/mL) was added to cultures on day 8 or day 10 and maintained in culture until day 14 to test the effect of this cytokine on inducing accelerated maturation of eosinophils (CCR3 low to CCR3 hi mature eosinophils). Control received no IL-7. Cells were stained gated for SSChi, live, and CD11b and shown for Siglec-F and CCR3 expression on days 10, 12, 14.

### Statistical Analysis

Student's t-test was used for two groups continuous variable comparisons while the Mann-Whitney U test was used for categorical variable comparisons. ANOVA test was used for multiple groups variable comparisons. All tests were two-tailed, and a P value of <0.05 was considered significant. Kaplan-Meier analysis and log-rank test were used to determine overall survival differences. Data visualization in all figures was accomplished by GraphPad Prism 10.2.0. Data are shown as means±SEM. Differences were considered significant at p<0.05.

### Example 2

### Results

### Pulmonary Resection Induces Eosinophil Activation and Systemic Eosinophilia

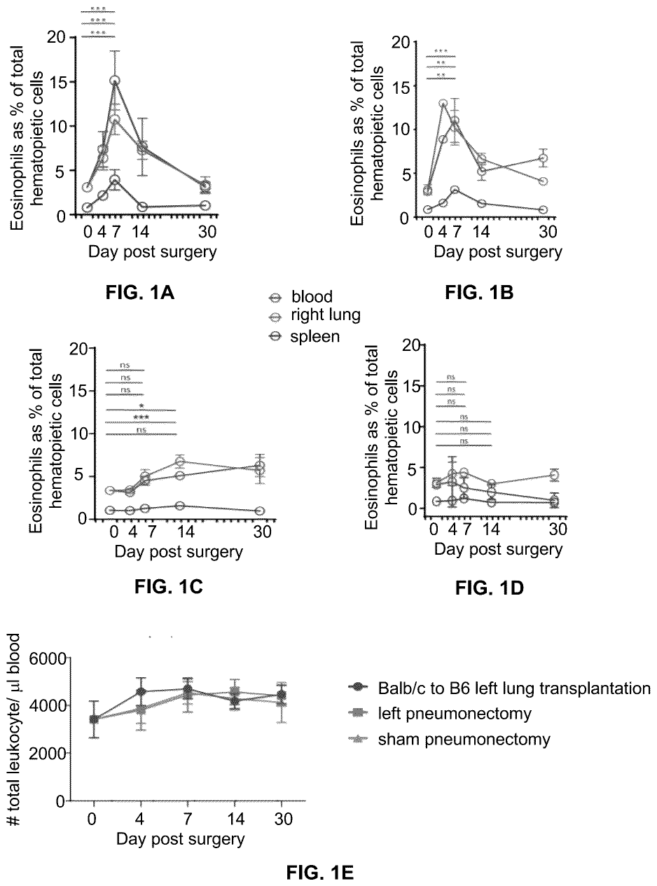

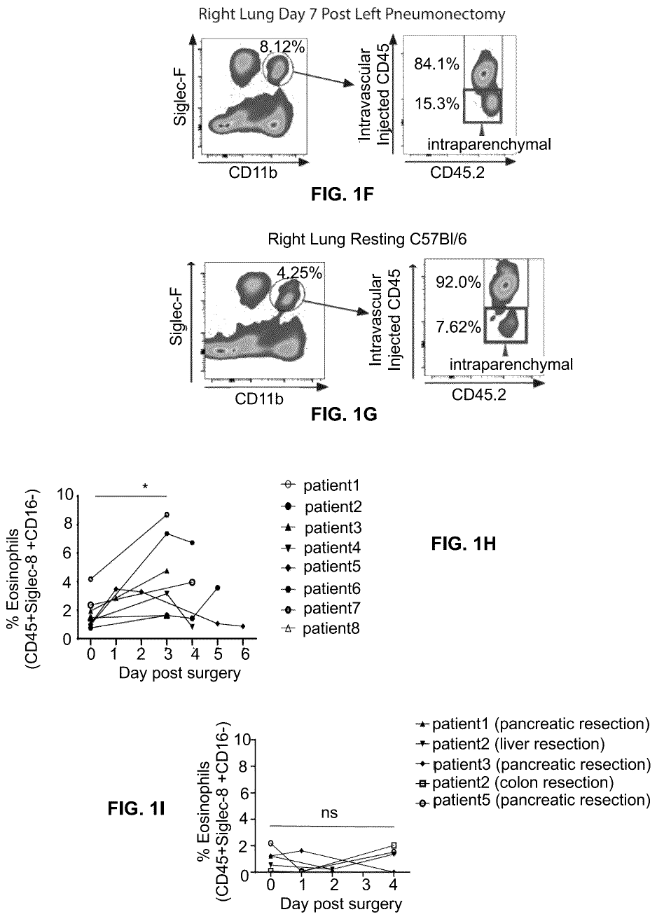

In humans, the right and left lungs contribute ≈53% and ≈47% of the pulmonary mass, respectively (15). In mice, however, the left lung consists of a single lobe and comprises only ≈30% of the total pulmonary mass (16). Thus, the resection of the left lung in the mouse is generally well tolerated while the resection of the right lung carries high morbidity, which was presumed to result from the removal of a large portion of the overall pulmonary volume (17). The possibility that systemic inflammation may be a contributing factor to poor recovery after lung resection was also considered (14). Leukocytes in the blood, right lung, or spleen were thus quantitated after either resection or transplantation of the left lung in the mouse. An early and significant increase in eosinophils in these compartments after lung resection or transplantation (FIGS. 1A-1B) was noted. Only a minimal and delayed increase in eosinophils was noted after sham thoracotomy, while abdominal operations like pancreatectomy did not lead to any substantial change in eosinophils (FIGS. 1C-1D). Thus, the increase in eosinophils was the result of pulmonary resection rather than incision into the thoracic or abdominal cavity. Such an increase in the relative number of eosinophils occurred in the absence of significant leukocytosis (FIG. 1E) or T cell, NK cell, B cell, or myeloid cell expansion in the blood or peripheral tissues. Furthermore, eosinophil extravasation and infiltration into the pulmonary parenchyma was increased in the right lung after left lung resection (FIGS. 1F-1G). The peripheral blood of patients undergoing lung resection was examined for a variety of malignant and benign conditions. It was noted that, similar to mice, circulating eosinophils increased early after lung resection but not following abdominal operations (FIGS. 1H-11). Similar to mice, other leukocytes such as T and B cells did not increase in the blood after lung surgery in humans. Taken together, this data demonstrates that lung resection triggers eosinophilia in both mice and humans.

### Pulmonary Resection Induces Activation of Lung Eosinophils and Accelerates Maturation of Eosinophils in the Bone Marrow

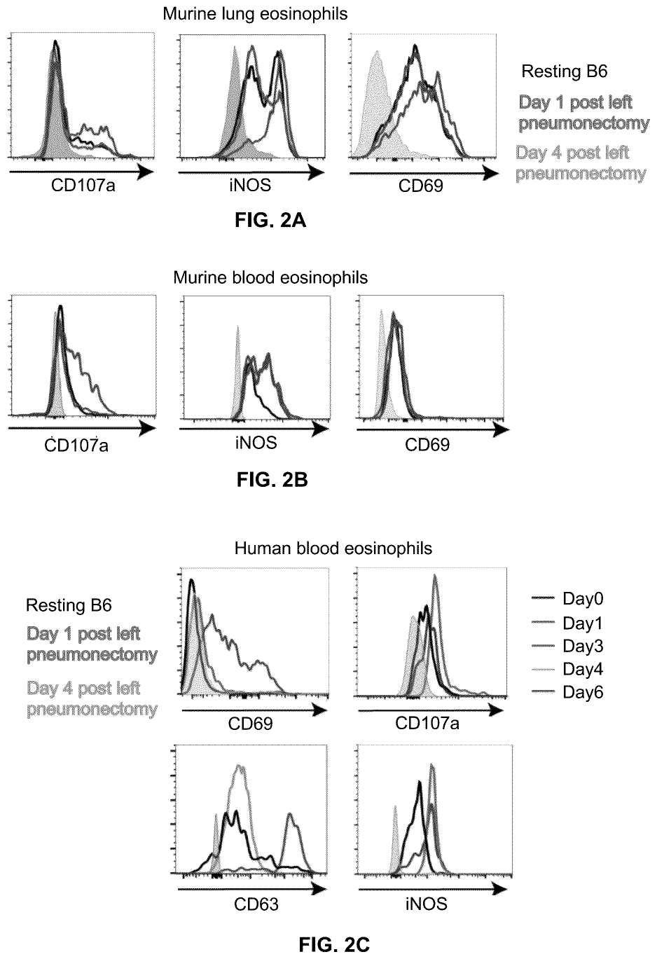

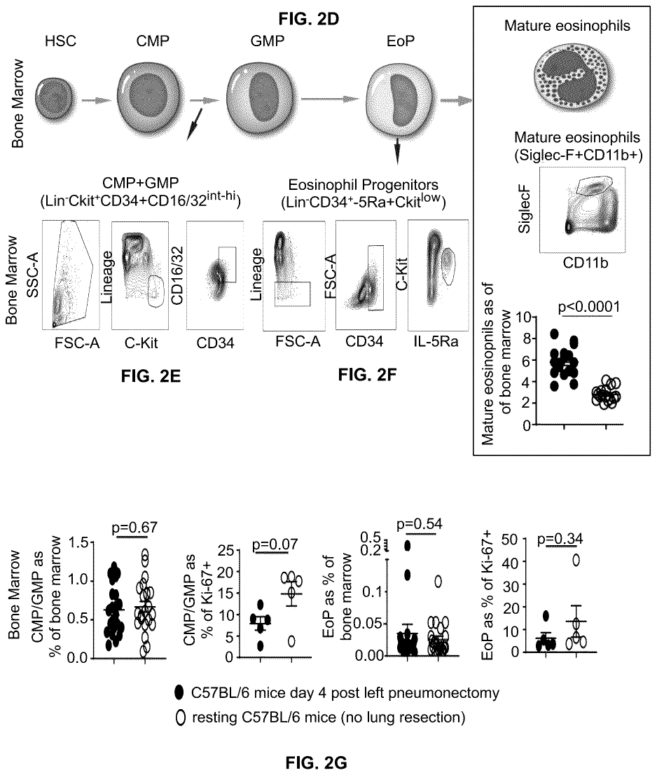

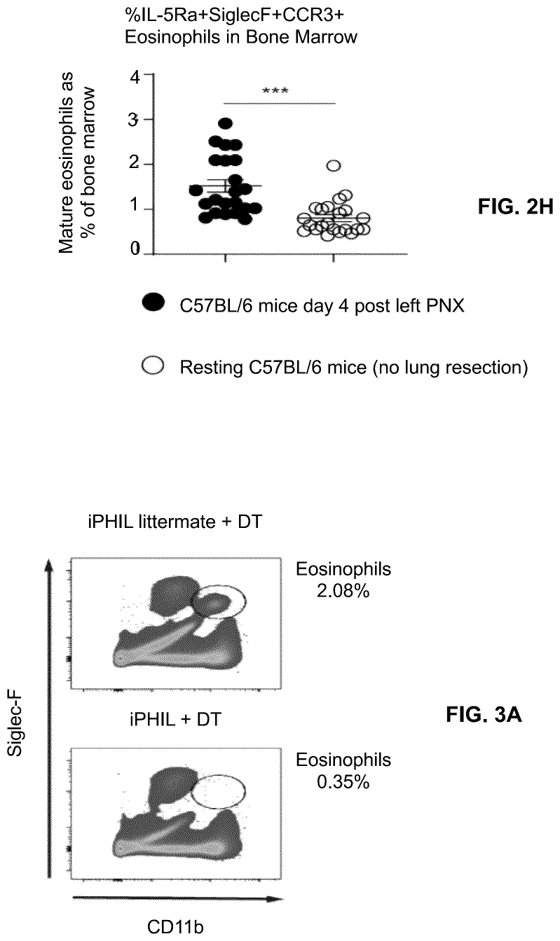

Markers of activation on eosinophils were examined in the right lung and blood after left pneumonectomy (PNX) in mice. Within one day (≈18 hours post resection) lung eosinophils demonstrated signs of activation, such as upregulation of CD69 increased degranulation, as measured by surface CD107a, and an increase in metabolic activity as measured by upregulation of iNOS (FIG. 2A). Blood eosinophils also showed some, albeit lower, level of activation (FIG. 2B). Activation of blood eosinophils was also evident in humans after lung resection (FIG. 2C).

Based on published data by Hellings and colleagues the possibility that lung resection may disinhibit eosinophils by decreasing the levels of local inhibitory receptors was initially considered (18). Surprisingly, exactly the opposite finding was noted with higher levels of multiple inhibitory receptors, co-stimulatory receptors, as well as major histocompatibility class II molecules on multiple cell types in the remaining right lung post left pneumonectomy. It is thus unlikely that surgical resection simply disinhibits eosinophil activation and infiltration due to loss of inhibitory receptors.

Next, it was determined if the eosinophilia after pulmonary resection was due to increased production in the bone marrow or mobilization from alternative sites. Eosinophil development in the murine bone marrow follows a defined pathway of maturation from hematopoietic stem cell (HSC) to common myeloid progenitor (CMP) to granulocyte/monocyte progenitor (GMP) to eosinophil lineage-committed progenitor (EoP) to mature eosinophil (19) (FIGS. 2D-2G). Evaluation of bone marrow from either resting or post left pneumonectomy mice demonstrated no differences in CMPs/GMPs or EoPs, but rather demonstrated an increase in mature eosinophils, defined by either Siglec-F+CD11b⁺ (FIGS. 2D-2G) or IL-5Rα⁺Siglec-F⁺CCR3⁺ (FIG. 2H) expression by day four post-lung resection (20). Such data suggested that the increase in eosinophils after lung resection occurs due to accelerated maturation from EoPs rather than increased levels of proliferation of progenitor cells. Extramedullary eosinopoiesis in the lung has been reported at times of inflammation-associated stress (21). However, no differences in CMPs/GMPs or EoPs in the right lung after left pneumonectomy when compared to resting lungs were detected. Thus, the data suggests that lung resection contributes to both the activation of lung eosinophils as well as their accelerated development in the bone marrow.

Eosinophils Play a Deleterious Role in Recovery after Major Lung Resection

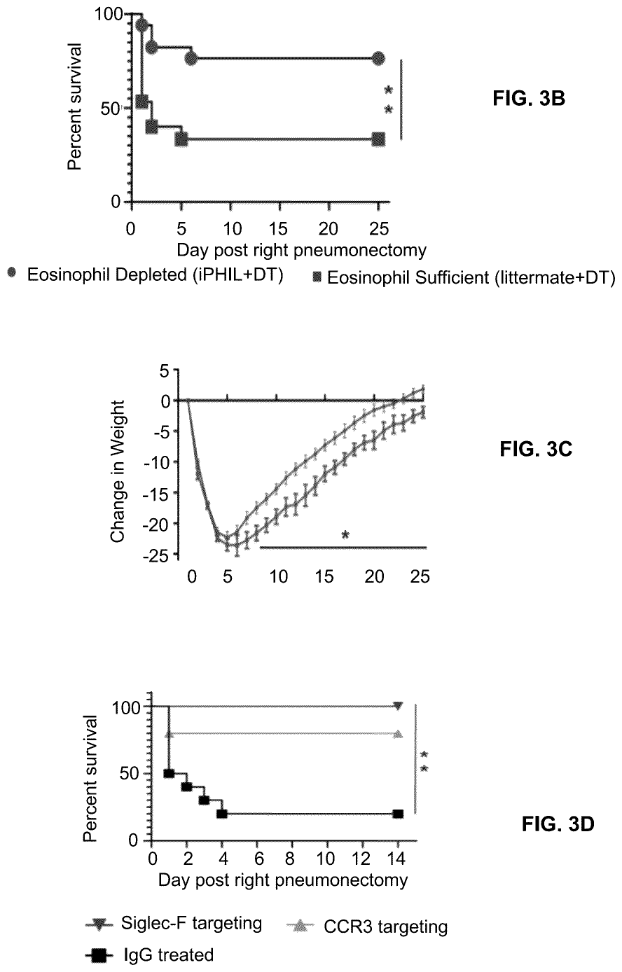

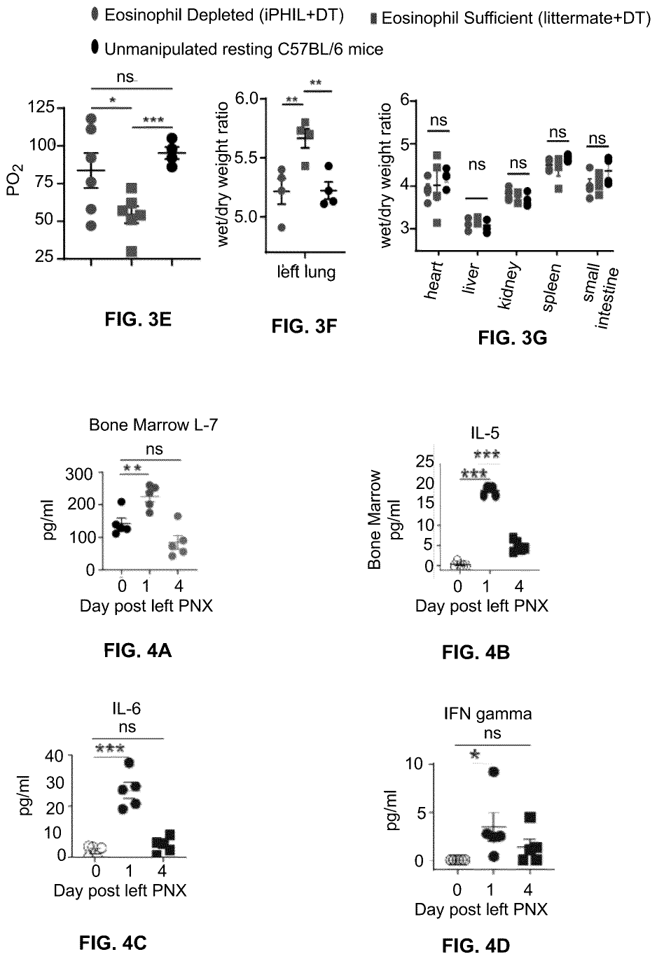

Unlike resection of the left lung, a right pneumonectomy is especially poorly tolerated in mice presumably due to the removal of substantial amount of pulmonary tissue (17). To evaluate if eosinophils influence postoperative recovery after major pulmonary resection, right pneumonectomies' were performed in the conditional eosinophil-deficient strain of mice (iPHIL) where the human diphtheria toxin (DT) receptor is expressed under the control of the endogenous eosinophil peroxidase genomic locus (22) (FIG. 3A). Unlike wild-type littermates, where DT treatment did not affect eosinophil numbers, eosinophil depletion in PHIL mice improved survival and recovery, as measured by weight gain (FIGS. 3B-3C).

To explore eosinophil targeting using clinically relevant protocols right pneumonectomies were performed in mice treated with anti-CCR3 and anti-Siglec F antagonistic and depleting antibodies. Such protocols mirror clinical trials targeting these two pathways for eosinophil mediated diseases such as asthma, eosinophilic bronchitis as well as eosinophil gastritis and duodenitis (Siglec-8 is the human homolog to murine Siglec-F) (23-25). As can be evidenced eosinophil targeting utilizing pathway improved survival after right pneumonectomy over age matched IgG control-treated mice (FIG. 3D). Eosinophil depletion also improved oxygenation and pulmonary edema to levels similar to wild type resting mice (FIGS. 3E-3F) without affecting fluid accumulation in other tissues (FIG. 3G). Interestingly, all morbidity and mortality occurred within a few days of resection and animals surviving past day five remained alive long-term. Thus, eosinophils play a deleterious role after lung resection by contributing to increases in both morbidity and mortality.

### IL-7 Signaling Plays a Critical Role in Stress-Induced Eosinophil Maturation in the Bone Marrow

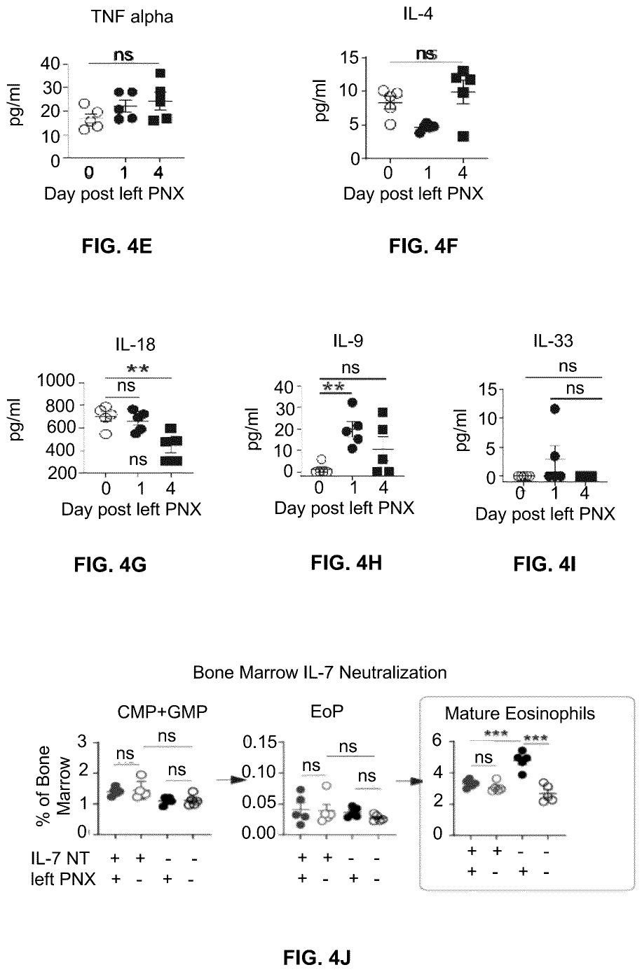

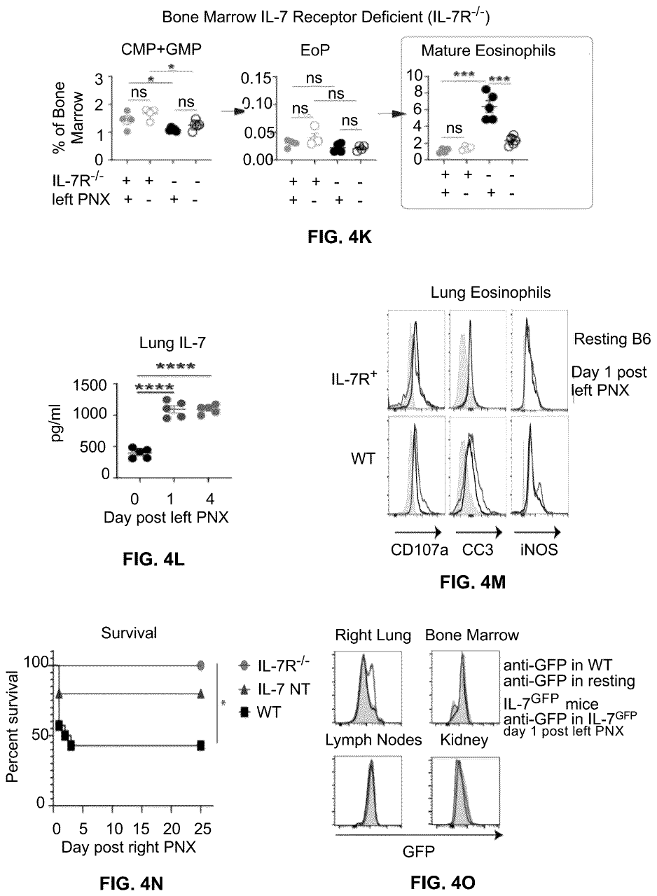

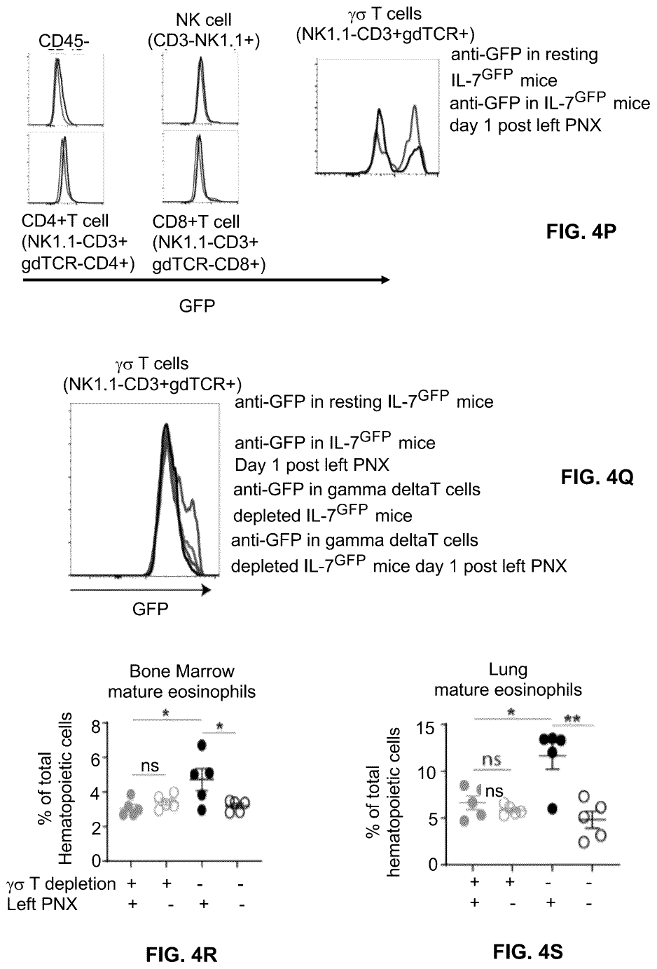

EoPs comprise approximately 0.05% of lineage negative CD34⁺ cells in the bone marrow and increase significantly in response to signals induced by asthma or helminth infection (26). While IL-3, GM-CSF, and IL-5 all belong to the same p common chain cytokine family, IL-5 is the only eosinophil hematopoietic cytokine in mice and humans with the ability to generate EoP (27-30). However, the mechanisms promoting the final steps of eosinophil maturation from the EoP to the mature eosinophil stage, a process that is enhanced after lung resection (FIG. 2D), are poorly defined. Cytokine levels in the bone marrow on days 1 and 4 post left pneumonectomy were evaluated next. Of cytokines known to influence eosinophil biology or general inflammation, only IL-7 (FIG. 4A) and IL-5, IL-6, IFN-γ and IL-9 (FIGS. 4B-41) concentrations were increased on day 1.

The bone marrow of mice after left pneumonectomy was evaluated in the presence or absence of IL-5, IL-7, IL-9 neutralization or IL-6 receptor blockade. IL-5 neutralization blunted EoP development, but stress-induced eosinophil maturation still occurred with an increase in mature eosinophils compared to non-resection control mice. IL-9 or IL-6 neutralization/blockade did not affect any step of eosinophil development compared to IgG-treated controls. Conversely, IL-7 neutralization resulted in a failure for cells to undergo stress-induced maturation while CMP/GMP or EoP populations were not affected (FIG. 4J). Stress-induced eosinophil maturation in the bone marrow after left pneumonectomy was also abrogated in IL-7 receptor deficient mice (IL-7R^{-/-}, also known as IL-7Ra^{-/-}) (FIG. 4K). Controversy exists whether eosinophils can directly respond to IL-7 (34, 35). When adding IL-7 to cultures of EoPs, no increase in eosinophil maturation compared to saline control conditions was noted.

Similar to bone marrow, IL-7 levels increased in the right lung after left pneumonectomy and eosinophil activation was evident in the right lung as measured by CD107a, iNOS and CCR3 upregulation (FIGS. 4L-4M). Consistent with these data elimination of IL-7 signaling, either through the use of IL7R^{-/-} mice or antibody-mediated neutralization, improved survival after right pneumonectomy (FIG. 4N). Through the use of IL-7^{GFP} reporter mice increased production of this cytokine in the remaining right lung after left pneumonectomy was detected (FIG. 4O). IL-7-producing cells were not detected in the bone marrow, mediastinal lymph nodes or other systemic organs such as the kidney after lung resection.

The source of IL-7 was evaluated using the aforementioned IL-7^{GFP} reporter mice. Evaluating lung digests it was noticed that the biggest change in the expression of IL-7 occurred in γδ T cells (FIG. 4P). To validate this further γδ T cells were depleted from mice prior to resection based on established methodology (36) and noted that upregulation of IL-7 levels was abated by flow cytometry (FIG. 4Q). In addition, depletion of γδ T cells ameliorated eosinophilia associated with lung resection (FIGS. 4R-4S). Taken together this demonstrates that lung surgery leads to elaboration of the cytokine IL-7 from γδ T cells which promotes stress-induced eosinophil activation in the lung, maturation in the bone marrow, and, perhaps more importantly, mediates the deleterious effects of eosinophils after lung resection.

### IL-7 Signaling in ILC2s Plays a Critical Role in Stress-Induced Eosinophil Maturation and Activation of Lung Eosinophils

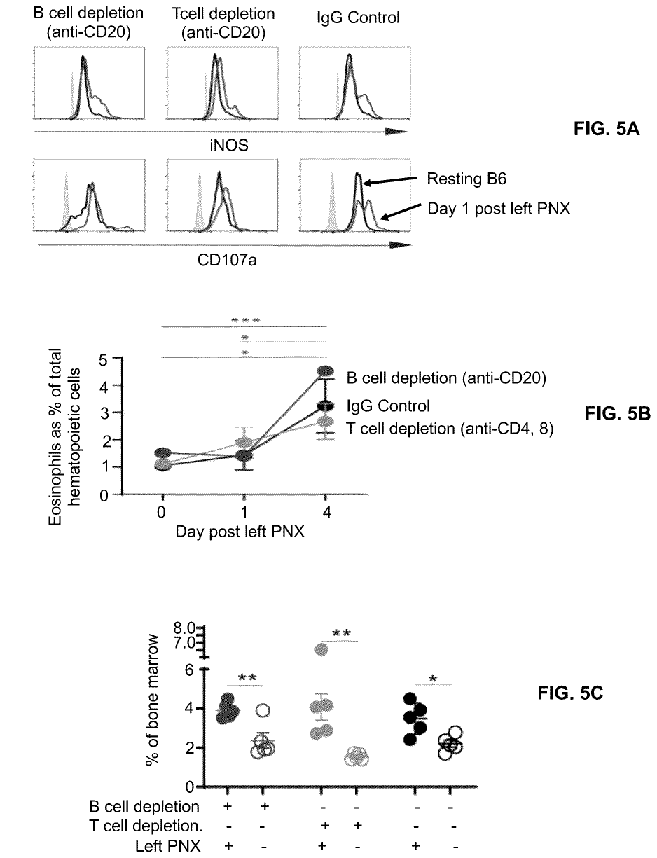

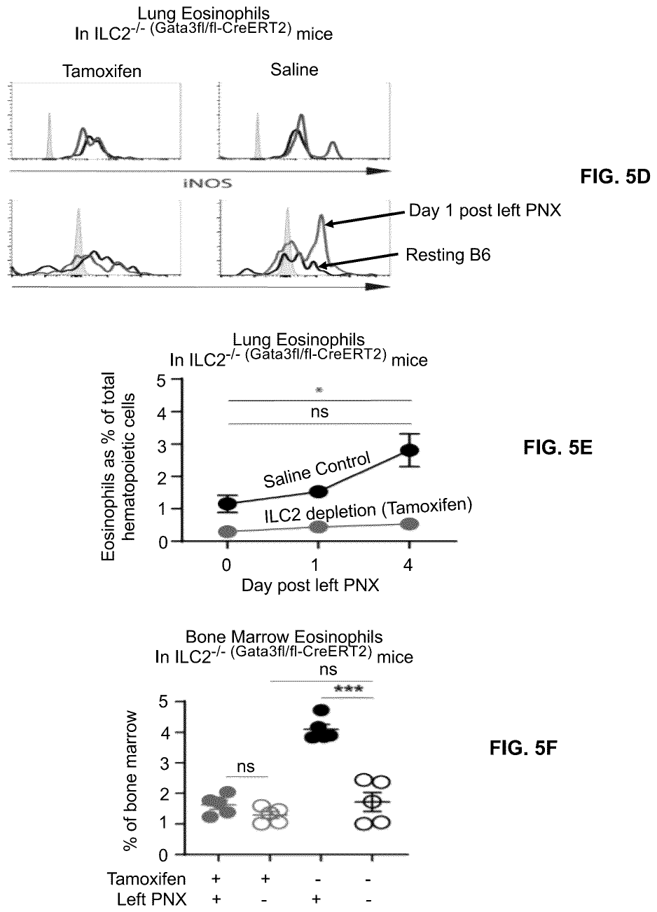

As IL-7 did not directly affect the activation and maturation of eosinophils, cellular mechanisms linking this cytokine to eosinophil activation were explored. As IL-7 is a well-described growth and survival factor for T cells, B cells and ILCs (37, 38). Thus, cell specific depletion strategies were utilized to determine if any of these cell populations could directly affect eosinophil activation. Neither T nor B cell depletion affected eosinophil activation in the lung (as measured by CD107a and iNOS expression) (FIG. 5A), quantitative expansion in the lung (FIG. 5B) or stress-induced eosinophil maturation in the bone marrow (FIG. 5C). By contrast, depletion of ILC2s through administration of tamoxifen to Gata3^{fl/fl}-CreERT2 mutant mice (39) abrogated stress-induced eosinophil maturation in the bone marrow and eosinophil activation and expansion in the lung (FIGS. 5D-5F) without affecting other stages of eosinopoiesis. Collectively, these experiments suggest that IL-7 production after pulmonary resection mediates deleterious effects through the activation of eosinophils in an ILC2-dependent fashion.

### IL-7 Mediated Modulation of ILC2s Contributes to Stress-Induced Eosinophil Maturation and Activation in a GM-CSF Dependent Fashion

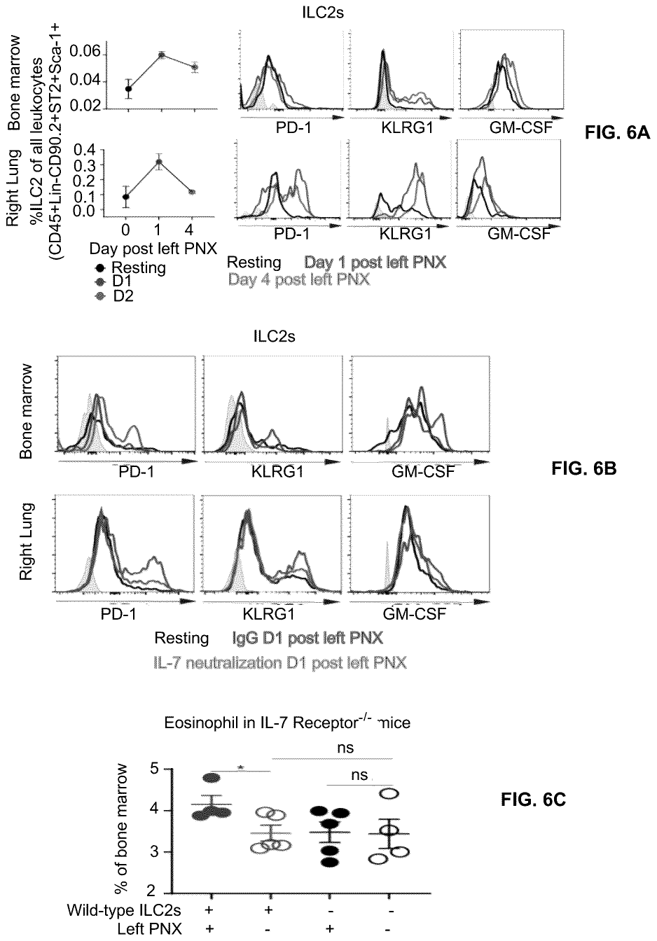

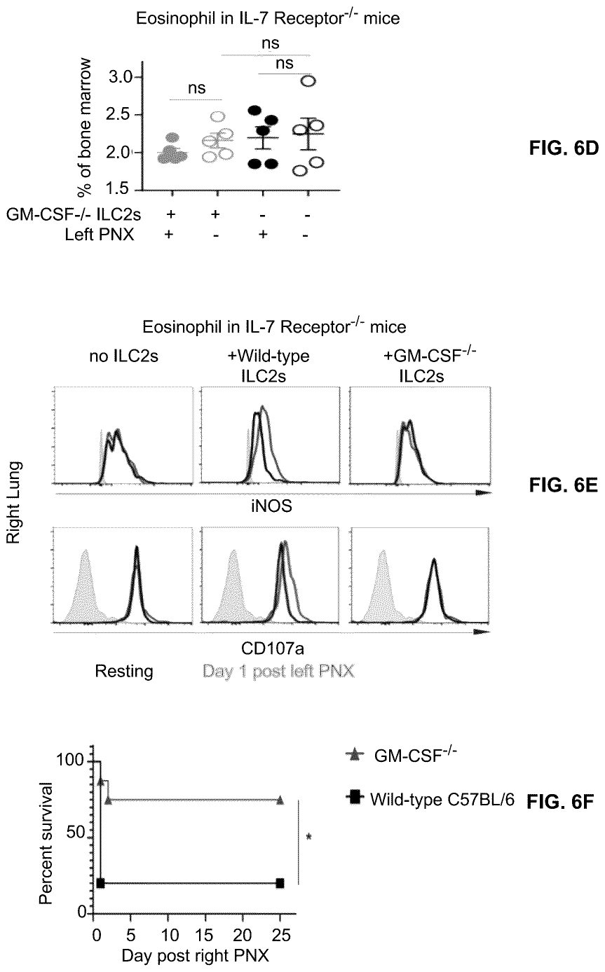

While IL-33 has been demonstrated to play a critical role in the activation of ILC2s (40, 41), no discernible shift in the expression levels of this cytokine after lung resection was able to be detected. IL-7, while considered a cytokine important for ILC2s survival, has been demonstrated to play a role in their activation as well (42). ILC2s numbers and their activation state was measured in the bone marrow and right lung after left lung resection. ILC2s expressed canonical markers of activation, such as KLRG1 and PD-1, with higher levels in the lung compared to the bone marrow (43, 44) (FIG. 6A). PD-1 expression of ILC2s has been previously correlated with GM-CSF production by this cell population (45) and increased GM-CSF expression in ILC2s in the bone marrow and lung after pulmonary resection were also observed (FIG. 6A). To further link pneumonectomy-mediated cytokine elaboration to ILC2 activation a one-time dose of IL-7 neutralizing antibody was administered to mice at the time of left pneumonectomy. It was noted that ILC2 activation, as measured by PD-1, KLRG-1 and GM-CSF levels, was ameliorated in the presence of IL-7 blockade on post-operative day number one (FIG. 6B). Such data thus links lung resection-elaborated IL-7 to ILC2 activation.

While IL-5 production by ILC2s can alter eosinophil physiology (40, 46, 47), the data did not implicate IL-5 in post-lung resection stress-induced eosinophil maturation and activation. GM-CSF is a monomeric glycoprotein cytokine that has been linked to eosinophil survival and activation in models of colitis (48), allergic asthma (49) and tumor immunity (50). While activated ILC2s produce GM-CSF (45, 51, 52), their contribution to effector functions of eosinophils is poorly defined. To examine the importance of ILC2-derived GM-CSF in eosinophil responses after lung resection, adoptive transfer studies of ILC2s into ILC2-deficient mice were completed. IL-7Ra^{-/-} mice were used as recipients since it was demonstrated that T and B cells did not play a critical role in mediating eosinophil effector functions following lung resection. To this end, adoptive transfer of ex vivo expanded wild-type, but not GM-CSF-deficient ILC2s, restored accelerated eosinophil maturation in the bone marrow without affecting other steps of eosinopoiesis (FIG. 6C-6D). Absent GM-CSF expression in ILC2s resulted in a reduced level of eosinophilic activation (iNOS, CD107a) in the lung (FIG. 6E). While some lung cytokines that could modulate eosinophils were elevated post-lung resection, the data suggest that GM-CSF elaborated from ILC2s is the key downstream mediator of detrimental eosinophils function in this situation. Consistent with this, GM-CSF^{-/-} mice survived a right pneumonectomy at a higher rate than wild-type mice (FIG. 6F). Taken together, it is demonstrated herein that GM-CSF production by ILC2s activated by IL-7 post-lung resection is required for eosinophil activation and subsequent pulmonary damage.

Pulmonary Toxicity Results from Eosinophil-Dependent iNOS-Mediated Damage

Despite the hypoxia, pulmonary edema, and mortality of eosinophil-sufficient mice after pulmonary resection, substantial cellular infiltration in the left lung following a right pneumonectomy was not observed. Thus, it is unlikely that eosinophil-mediated pulmonary damage post-pneumonectomy is mediated by the recruitment of other leukocytes. Since eosinophils can cause pulmonary damage due to the production of cytotoxic mediators, such as eosinophil-specific granule proteins (53), the possibility that eosinophils were directly killing stromal cells was considered next. Evaluation for cell death using TUNEL staining of remaining lung tissue, however, revealed almost no apoptosis in eosinophil-sufficient or deficient lungs. Thus, an alternative mechanism must exist by which eosinophils mediate damage to the lung.

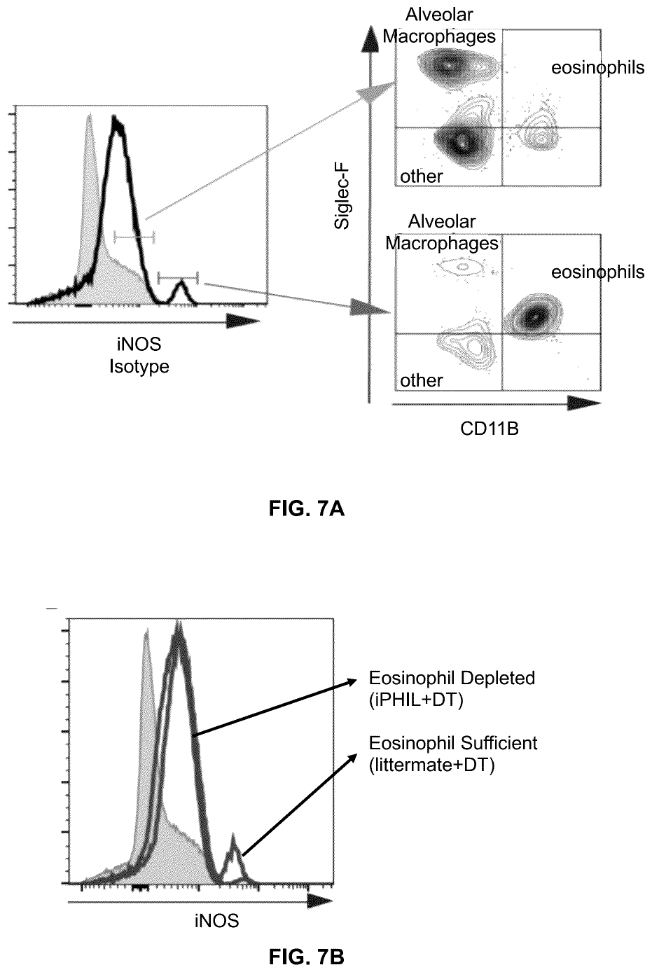

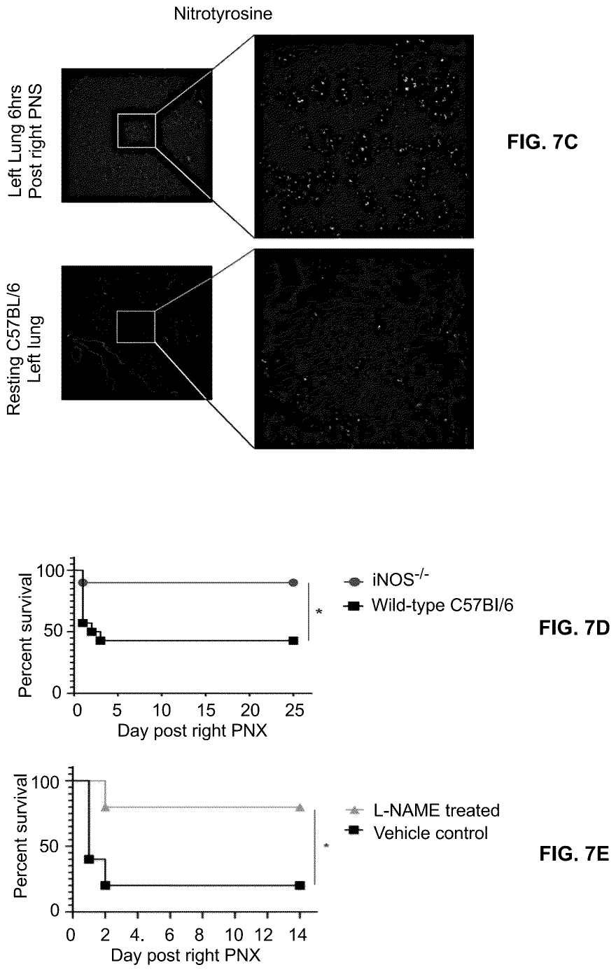

It was demonstrated that iNOS is increased in pulmonary eosinophils after lung transplantation (54, 55) and, as described above, the expression of this enzyme is increased following pulmonary resection as well. In addition, eosinophils are a major source of iNOS and a source of nitric oxide (NO)-mediated damage in severe asthma (56, 57). In the setting of acute lung injury, iNOS and NO have been shown to mediate deleterious effects through nitration or nitrosylation of key signaling intermediates, interference with surfactant production (58) and contribution to diffuse capillary leak resulting in pulmonary edema (59). Therefore, the expression of iNOS in the left lung post-right pneumonectomy was evaluated next and it was noted that the highest levels were predominantly expressed by Siglec-F+CD11 b⁺ eosinophils (FIG. 7A). Consistent with this, depletion of eosinophils resulted in loss of the iNOS^{high} population in the left lung after a right pneumonectomy (FIG. 7B). Histologic evaluation of the left lung six hours after the right pneumonectomy revealed products of nitrosylation with extensive nitrotyrosine staining (FIG. 7C). Consistent with this iNOS knockout mice had improved survival after a right lung pneumonectomy compared to wild-type controls (FIG. 7D). More importantly, treatment with the clinically utilized NOS inhibitor N(gamma)-nitro-L-arginine methyl ester (L-NAME) (60) in the perioperative period similarly improved survival over control vehicle-treated mice (FIG. 7E).

### Example 3

Effects of Cytokines IL-5 and IL-5a after Lung Resection
IL-5 Neutralization Contributes to Accelerated Mortality after Lung Resection

Eosinophil activation after lung resection directly contributes to post-operative morbidity and mortality. Depletion of eosinophils, utilizing experimental but not clinically relevant strategies such as diphtheria toxin administration in genetically engineered mice, can improve post-operative survival and recovery. Multiple IL-5 targeting reagents are FDA-approved for atopic diseases. Specifically, FASENRA (benralizumab), an anti-IL-5receptor α (IL-5Rα) targeting antibody, rapidly and efficiently eliminates eosinophils for the treatment of atopic disease through the process of antibody-mediated cytotoxicity (ADCC). NUCALA (mepolizumab) and CINQAIR (reslizumab) destroy eosinophils by systemically neutralizing the cytokine IL-5.

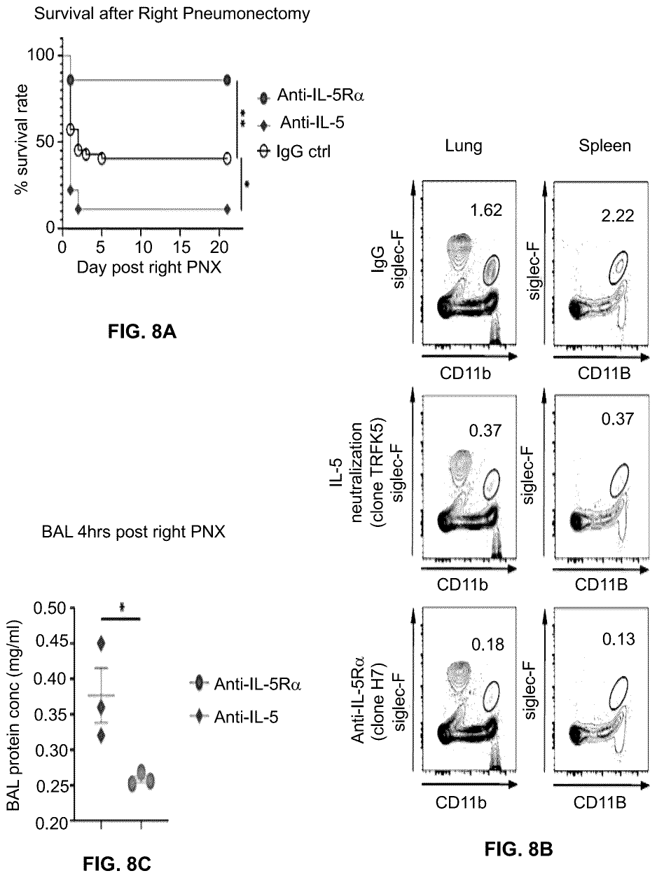

To repurpose such biologic therapy for improving recovery after lung resection, a right pneumonectomy (removing approximately 70% of total pulmonary mass) was performed in mice in the presence of a murine analog of FASENRA, which depletes IL-5Rα expressing cells via ADCC, (clone H7) (104) or an IL-5 neutralizing antibody (clone TRFK5) (105). While, mice treated with control IgG demonstrated an approximately 50% survival after such a major lung resection, depletion of eosinophils utilizing the anti-IL-5Rα ADCC-mediated strategy improved survival to near 100% (FIG. 8A). Neutralization of IL-5, however, resulted in rapid and complete demise of nearly all mice within 12-24 hours post-resection (FIG. 8A). Such mortality occurred despite efficient eosinophil depletion by both IL-5 neutralization and anti-IL-5Rα ADCC (FIG. 8B). Unlike mice treated with anti-IL-5Rα depleting antibody, the bronchioalveolar lavage (BAL) of remining left lung of mice treated with an IL-5 neutralizing antibody demonstrated high protein content, suggestive of diffuse alveolar damage (FIG. 8C). Such data suggested a more complicated role for IL-5 in controlling lung homeostasis after resection, rather than just depletion of eosinophils.

Lung Resection Mediates a Rapid Migration of both IL-5Rα+ and IL-5Rα⁻ Neutrophils to Remaining Pulmonary Tissue

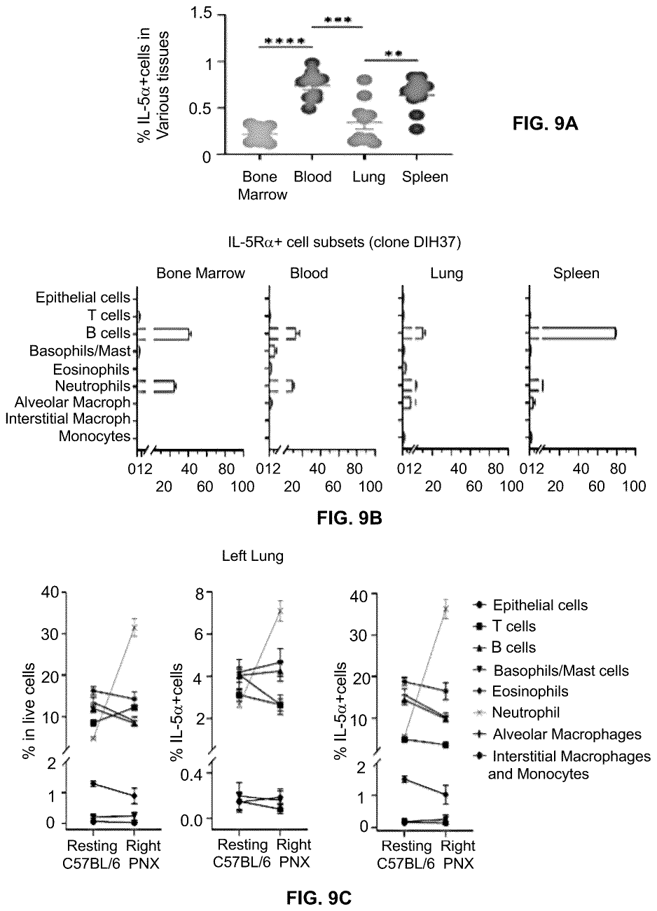

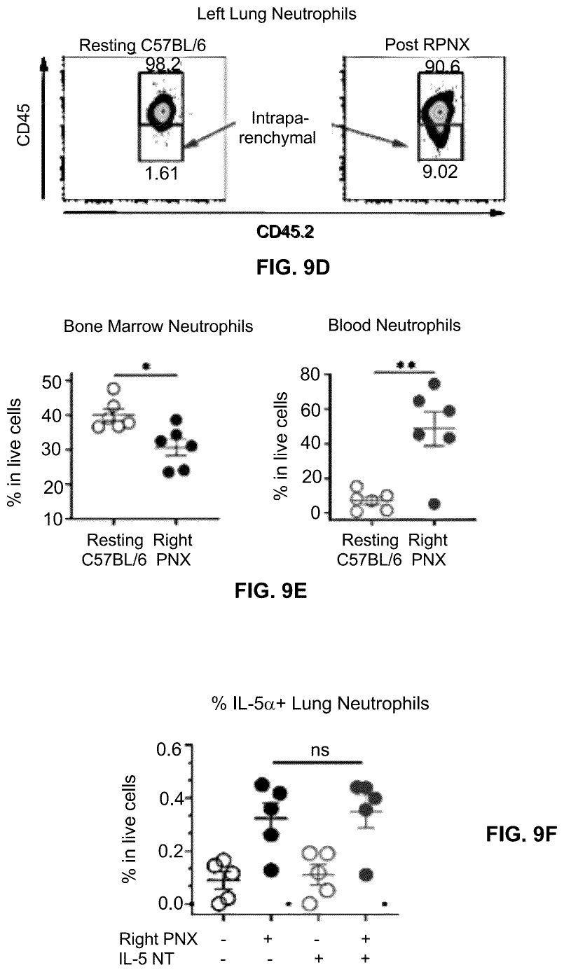

The expression the IL-5 receptor on various cell types in the lung, bone marrow, blood, and spleen was examined. Utilizing clone DIH37, which has been demonstrated to represent sensitive and specific staining of the IL-5Rα chain on multiple murine cell populations (106), it was noted that approximately 1% of cells expressed this receptor at rest (FIG. 9A). Expression occurred broadly across multiple cell types including B cells, neutrophils, and monocytes/macrophages as well as eosinophils (FIG. 9B). While it is well described that eosinophils constitutively express IL-5Rα (107) they represent a rare cell population in the lung (comprising less than 5% of lung-resident cells) and thus were not the dominant cell type expressing this receptor.

Early changes in cellular composition of the left lung after right pneumonectomy (R PNX) were next evaluated focusing on both IL-5Rα+ and IL-5Rα⁻ cellular subsets. Cellular changes four hours after resection were evaluated based on the rapid animal demise evident after IL-5 neutralization (FIG. 8A). The only major change in cell numbers occurred in CD45⁺ CD11c⁻CD11 b⁺ EPX⁻SiglecF⁻Ly6G⁺ neutrophils, a cellular subset phenotypically different from CD45⁺ CD11c⁻CD11b⁺ SiglecF⁺ EPX⁺ Ly6G⁻eosinophils (FIG. 9C). Such an increase was evident for both IL-5Rα⁺ and IL-5Rα⁻neutrophils (FIG. 9C). Neutrophils not only increased in number but also migrated into the pulmonary parenchyma of the remining left lung after right pneumonectomy (FIG. 9D). Concurrent evaluation of the bone marrow and blood demonstrated a decrease of this cell population in the bone marrow and an increase in the blood stream, suggesting rapid migration of this cell population from the bone marrow to the remaining lung within hours after resection (FIG. 9E). Lung resection, but not IL-5 neutralization in and of itself, contributed to such migratory patterns (FIG. 9F). Thus, the role of IL-5 in controlling neutrophil biology remained unclear.

### IL-5 Ameliorates G-CSF Mediated Activation of IL5Rα+Neutrophils

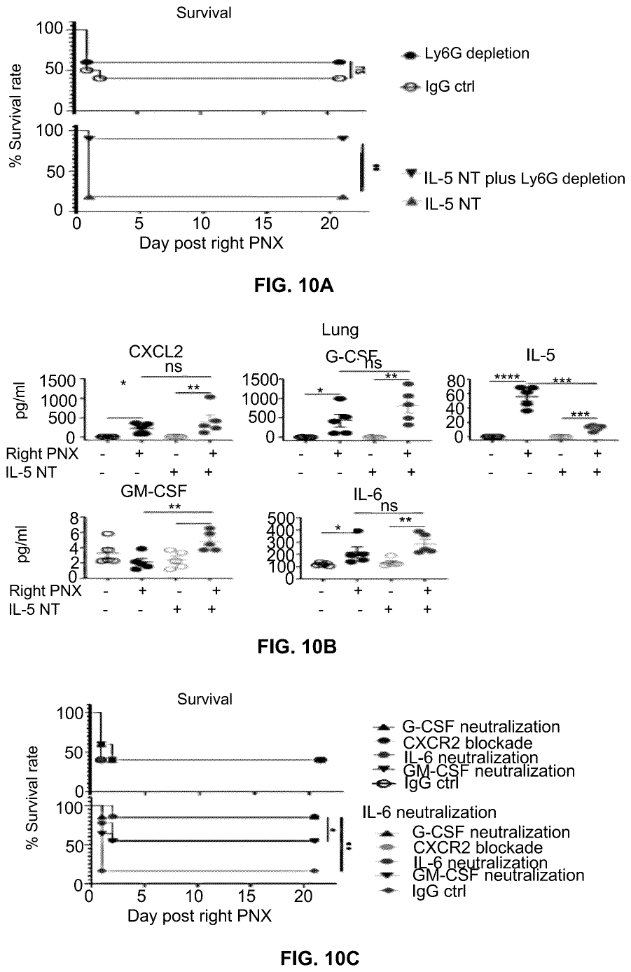

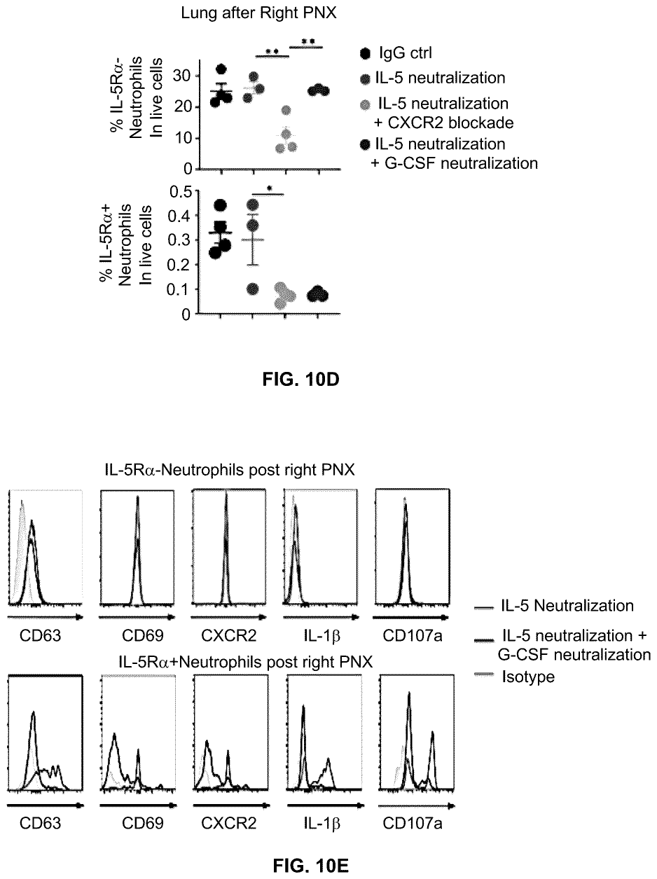

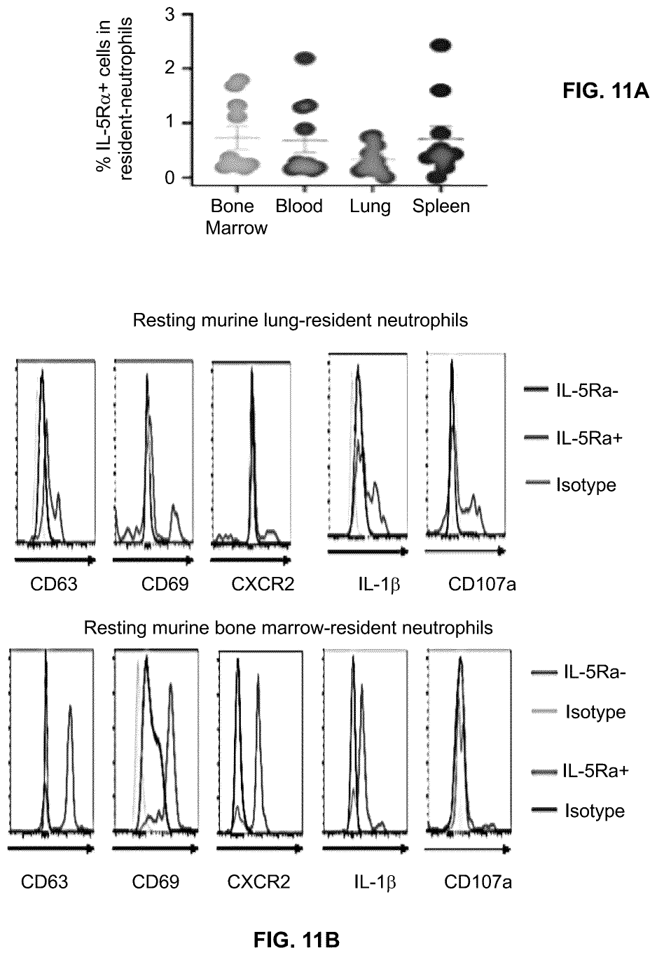

Neutrophils were depleted with the systemic administration of anti-Ly6G (clone 1A8) (108) prior to lung resection. There were no differences in survival between neutrophil sufficient and deficient recipients after right pneumonectomy (FIG. 10A, top). After IL-5 depletion, however, stark differences were evident between survival of neutrophil deficient and sufficient animals. While rapid and substantial mortality was evident in IL-5 depleted neutrophil sufficient mice, depletion of neutrophils resulted in near complete survival after lung resection (FIG. 10A, bottom). This seemed to suggest that in the absence of IL-5 neutrophils that express IL-5R take on a pathogenic role that may equal, or even surpass, toxicity mediated by eosinophils.

Whether IL-5 deficiency may alter neutrophil function by affecting the chemokine and cytokine milieu of remining lung tissue was examined. Analysis of the left lungs four hours after right pneumonectomy did demonstrate upregulation of select mediators of neutrophil migration and function such GM-CSF, IL-6, G-CSF and CXCL2 (FIG. 10B). However, levels of all but GM-CSF were affected more by lung resection rather than IL-5 neutralization. Such cytokines and chemokines were next targeted using either depleting antibodies or competitive inhibitors and it was noted that, similar to the neutrophil depletion experiments described above, no difference in survival was evident compared to control mice in the presence of IL-5 (FIG. 10C, top). However, in IL-5 depleted mice both G-CSF neutralization and blockade of the CXCL2/CXCR2 pathway substantially improved survival post-lung resection (FIG. 10C, bottom).

The remaining left lung post-right pneumonectomy in IL-5 depleted mice treated with either an CXCR2 antagonist or G-CSF neutralization was evaluated. Blockade of the CXCL2/CXCR2 axis decreased the number of both IL5Rα+ and IL5Rα⁻ neutrophils while G-CSF neutralization quantitatively altered only the IL5Rα+neutrophils (FIG. 10D). Furthermore IL-5 neutralization facilitated G-CSF mediated activation of IL5Rα+(but not IL5Rα⁻) neutrophils after lung resection (FIG. 10E). Taken together the present invention demonstrated that immediately after lung resection local upregulation of chemokines and cytokines mediates neutrophilic infiltration into remaining lung tissue with amelioration of cytotoxicity of such infiltrating cells by IL-5.

### IL-5Rα⁺ is Expressed During Neutrophil Development

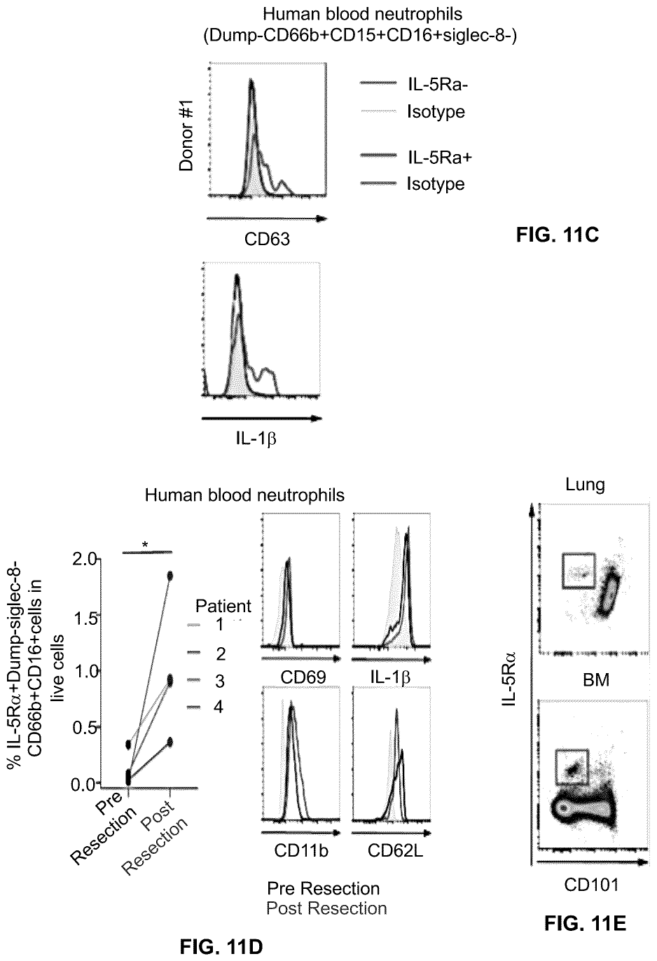

As IL-5Rα expression on neutrophils is poorly described, this cell population was characterized in both mice and man. While only a minority of neutrophils in peripheral organs express IL-5Rα (FIG. 11A), this cell population demonstrated higher expression of activation and cytotoxicity markers, even at baseline, in resting murine lungs (FIG. 11B) and bone marrow (FIG. 11B) when compared to IL-5Rα⁻ neutrophils. Similar to mice, IL-5Rα+neutrophils were present in the blood of healthy volunteers (FIG. 11C) and rapidly increased in patients undergoing lung resection (FIG. 11D). However, similar to IL-5 sufficient mice (not treated with IL-5 neutralizing antibody), no drastic change was evident in the activation of IL-5Rα-expressing neutrophils post-resection when compared to baseline (FIG. 11D). Such IL-5Rα+neutrophils demonstrated low expression of surface CD101, consistent with an immature phenotype (FIG. 11E). Thus IL-5Rα⁺ is expressed on immature, highly cytotoxic neutrophils which are “disinhibited” when the cytokine IL-5 is neutralized.

IL-5 Interferes with G-CSF Signaling in IL-5Rα+Neutrophils Through Alteration of JAK-2 and SOCS3

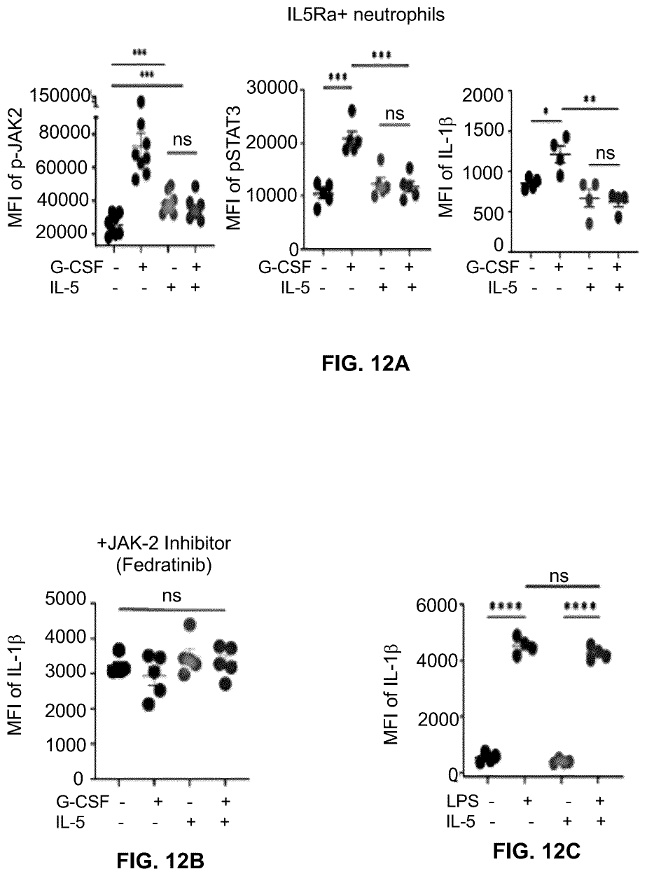

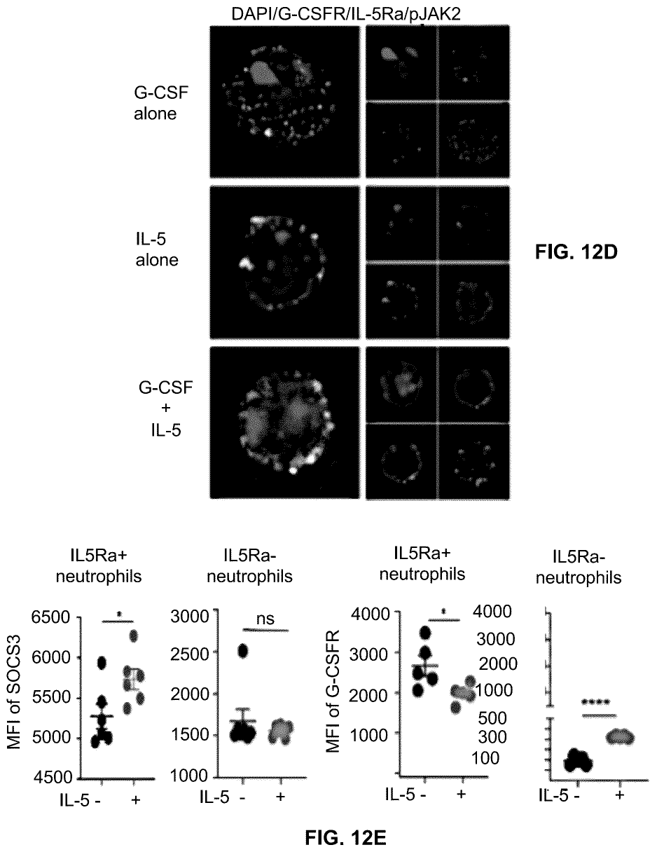

Signaling and activation of IL-5Rα+neutrophils were evaluated in vitro after cytokine supplementation. Canonic signaling of G-CSF occurs through the Janus kinase 2/Signal Transducer and Activator of Transcription 3 (Jak2/STAT3) pathway (109). Addition of G-CSF resulted in the activation of IL-5Rα+neutrophils, as measured by an increase of intracellular IL-1p, as well as the relative levels of phospho-JAK2 and phospho-STAT3, as determined by Phospho-flow staining in addition to the (FIG. 12A). Interestingly the concomitant addition of IL-5 to such cultures decreased the phosphorylation of JAK-2, STAT3, and production of IL-1p (FIG. 12A) supporting the notion that IL-5 ameliorates activation of IL-5Rα expressing neutrophils. No such inhibition was evident in IL-5Rα⁻ neutrophils. Such data supports the role of IL-5/IL-5Ra in ameliorating cytotoxicity of IL-5Rα expressing neutrophil subsets as described above. Since both G-CSF and IL-5 share the JAK-2 signaling intermediate we next inhibited this signaling moiety using the kinase inhibitor Fedratinib and noted complete inhibition of both G-CSF-mediated neutrophil activation as well as IL-5-mediated signaling (FIG. 12B). In addition, IL-5 could not inhibit neutrophil activation by lipopolysaccharide (LPS), which signals through the toll-like receptor 4/MyD88/TRIF pathway (FIG. 12C). Taken together such data suggests that one possible mechanism of IL-5 mediated inhibition of G-CSF signaling may involve alteration of JAK-2 mediated signaling.

To evaluate this further, a high resolution confocal microscopy of IL-5Rα+neutrophils in the presence of G-CSF, IL-5 or a combination of both cytokines was performed. In the presence of G-CSG phosphorylated JAK-2 (pJAK-2) there was a pattern of equal distribution along the cell membrane (FIG. 12D, left top). In the presence of IL-5, however, a substantial amount of pJAK-2 localized to patches of membrane containing IL-5Rα, a pattern that was recapitulated even when G-CSF was added to such cultures (FIG. 12D, left middle, bottom). Such data supports the notion that one mechanism for IL-5 mediated amelioration of G-CSF signaling may occur through the interference and availability of the JAK-2 signaling intermediate.

In addition to potential interference with JAK-2 mediated signaling IL-5 has been previously demonstrated to induce the expression of Suppressor of Cytokine Signaling 3 (SOCS3) in eosinophils (110). SOCS3 is a well-described negative regulator of G-CSF receptor (G-CSFR) expression and function. Similar to what has been described for eosinophils, IL-5Rα+neutrophils cultured with IL-5 upregulated SOCS3 and decreased surface expression of G-CSFR (FIG. 12E). Thus, several mechanisms exist to explain amelioration of IL-5Rα+neutrophil activation by IL-5.

Thus, the present invention demonstrates the efficacy of methods of eosinophil depletion that directly target and kill this cell population. Neutrophil progenitors are more cytotoxic than those in their more mature state. This is likely the result of developmental programs inherent to innate immunity, where destructive capacity is front loaded with control of such cytotoxicity imposed later during maturation. This is in direct contrast to adaptive immune responses where self-reactivity is generally eliminated by thymic selection before permitting lethal function. Nevertheless, the present invention demonstrates the efficacy of factors that directly deplete eosinophils such as benralizumab and in clinical use targeted eosinophil depletion, Siglec-8 targeting antibodies lirentelimab, and dexpramipexole which directly kills eosinophils.

The following references are cited herein:
1. Chen-Yoshikawa et al., Nagoya J Med Sci, 82:161-174 (2020).
2. Moffatt-Bruce, et al., J Thorac Cardiovasc Surg, 155:824-829 (2018).
3. Stephan et al., Chest, 118:1263-1270 (2000).
4. Heiden et al., J Thorac Cardiovasc Surg, 164:615-626 e613 (2022).
5. Ansari et al., Ann Thorac Surg 101:459-464 (2016).
6. O. M. Shapira and D. M. Shahian, Ann Thorac Surg, 56:190-195 (1993).
7. Mathru, et al., Chest, 98:1216-1218 (1990).
8. Gibbon, et al., Journal of Thoracic Surgery, 12:60-77 (1942).
9. Blanc et al., J Thorac Cardiovasc Surg, 156:1706-1714 e1705 (2018)
10. Parquin, et al., Eur J Cardiothorac Surg, 10:929-933 (1996).
11. Alvarez et al., J Thorac Cardiovasc Surg, 133:1439-1447 (2007).
12. Villeneuve, et al., Thorac Surg Clin, 16:223-234 (2006).
13. W. S. Turnage, Chest, 106:320-321 (1994).
14. Cerfolio et al., Ann Thorac Surg, 76:1029-1033 (2003).
15. Molina, et al., Am J Forensic Med Pathol, 33:368-372 (2012).
16. Thiesse et al., J Appl Physiol (1985) 109:1960-1968 (2010).
17. Li et al., J Thorac Cardiovasc Surg, 139:1637-1643 (2010).
18. Hellings et al., Eur J Immunol, 32:585-594 (2002).
19. H. Iwasaki et al., J Exp Med, 201:1891-1897 (2005).
20. Fulkerson, et al., J Immunol, 193:4043-4052 (2014).
21. Menzies-Gow et al., J Allergy Clin Immunol, 111:714-719 (2003).
22. Jacobsen et al., Allergy, 69:315-327 (2014).
23. Neighbour et al., Clin Exp Allergy, 44:508-516 (2014).
24. Imaoka et al., Clin Exp Allergy, 41:1740-1746 (2011).
25. Dellon et al., N Engl J Med, 383:1624-1634 (2020).
26. Abdala-Valencia et al., J Leukoc Biol, 104:95-108 (2018).
27. Martinez-Moczygemba, et al., J Allergy Clin Immunol, 112:653-666 (2003).
28. Lee et al., J Allergy Clin Immunol, 130:572-584 (2012).
29. Matthaei, et al., Mem Inst Oswaldo Cruz, 92 Suppl 2:63-68 (1997).
30. Schollaert, et al., PLoS One, 9:e116141 (2014).
31. Rosenberg, et al., Nat Rev Immunol, 13:9-22 (2013).
32. Gubernatorova et al., Front Immunol, 9:2718 (2018).
33. Louahed et al., Blood, 97:1035-1042 (2001).
34. Kelly et al., J Immunol, 182:1404-1410 (2009).
35. Cool, et al., Exp Hematol, 90:39-45 e33 (2020).
36. Chu et al., Mucosal Immunol, 7:1395-1404 (2014).
37. Vonarbourg, et al., Semin Immunol, 24:165-174 (2012).
38. Barata, et al., Nat Immunol, 20, 1584-1593 (2019).
39. Yagi et al., Immunity, 40:378-388 (2014)
40. Guo et al., Am J Transplant, 22:1963-1975 (2022).
41. Riedel et al., J Am Soc Nephrol, 28:2068-2080 (2017).
42. Takami et al., Int Immunol, (2023).
43. Olguin-Martinez, et al., Front Immunol, 12:757967 (2021).
44. Taylor et al., J Exp Med, 214:1663-1678 (2017).
45. Jacquelot et al., Nat Immunol 22:851-864 (2021).
46. Klein Wolterink et al., Eur J Immunol, 42:1106-1116 (2012).
47. Nussbaum et al., Nature, 502:245-248 (2013).
48. Griseri et al., Immunity, 43:187-199 (2015).
49. Nobs, et al., J Allergy Clin Immunol, 143:1513-1524 e1512 (2019).
50. Arnold et al., J Exp Med, 217, (2020).
51. Momiuchi et al., Int Immunol, 33:573-585 (2021).
52. Sudo et al., J Exp Med, 218, (2021).
53. Lynch et al., Transplantation, (2021).
54. Onyema et al., JCI Insight, 2, (2017).
55. Onyema et al., JCI Insight, 4, (2019).
56. Yamamoto et al., Clin Exp Allergy, 42:760-768 (2012).
57. Yousefi et al., J Leukoc Biol, 104:205-214 (2018).
58. Sittipunt et al., Am J Respir Crit Care Med 163:503-510 (2001).
59. Wang le et al., Am J Respir Crit Care Med, 165:1634-1639 (2002).
60. Cotter et al., Eur Heart J, 24:1287-1295 (2003).
61. Jacobsen et al., Annu Rev Immunol, 39:719-757 (2021).
62. Mitre, et al., Semin Immunopathol, 43:363-381 (2021).
63. Klion, et al., Annu Rev Pathol, 15:179-209 (2020).
64. LeMessurier, et al., Curr Allergy Asthma Rep, 19:36 (2019).
65. Mitra, et al., Redox Rep, 5:215-224 (2000).
66. Withers et al., Sci Rep, 7:44571 (2017).
67. Uderhardt et al., J Exp Med, 215:1003 (2018).
68. Gasteiger, et al., Science, 350:981-985 (2015).
69. Chojnacki et al., Commun Biol 2:181 (2019).
70. Barlow, et al., Annu Rev Physiol, 81:429-452 (2019).
71. LeSuer et al., J Allergy Clin Immunol, 152:469-485 e410 (2023).
72. Kopf et al., Immunity, 4:5-24 (1996).
73. Hiraki et al., Semin Intervent Radiol, 30:169-175 (2013).
74. Thompson, et al., Transl Lung Cancer Res, 8:48-57 (2019).
75. Kouro, et al., Int Immunol, 21:1303-1309 (2009).
76. Ortega et al., N Engl J Med, 371:1198-1207 (2014).
77. Haldar et al., N Engl J Med, 360:973-984 (2009).
78. Flood-Page, et al., Am J Respir Crit Care Med, 167:199-204 (2003).
79. Alpdogan, et al., Trends Immunol, 26:56-64 (2005).
80. de Saint-Vis et al., J Immunol, 160:1666-1676 (1998).
81. Kroncke, et al., Eur J Immunol, 26:2541-2544 (1996).
82. Sorg, et al., Immunobiology, 198:514-526 (1998).
83. Soslau, et al., Cytokine, 9:405-411 (1997).
84. Duchesne, et al., Front Immunol, 13:975914 (2022).
85. Simon et al., Am J Physiol Lung Cell Mol Physiol, 291:L851-861 (2006).
86. Pazdrak, et al., J Immunol, 186:6485-6496 (2011).
87. Chen, et al., Front Immunol, 12:747324 (2021).
88. Kreisel et al., Blood, 118:6172-6182 (2011).
89. Lazarus, et al., Acta Haematol, 144:355-359 (2021).
90. Diamant et al., Allergy, 74:1835-1851 (2019).
91. Ricciardolo, et al., Physiol Rev 84:731-765 (2004).
92. Golden, et al., Front Pharmacol, 12:761496 (2021).
93. Munoz-Caro et al., Parasit Vectors, 8:607 (2015).
94. Ribot, et al., Nat Rev Immunol, 21:221-232 (2021).
95. Graulich et al., Pediatr Res, 48:679-684 (2000).
96. Mc et al., Lab Invest, 90:128-139 (2010).
97. Siddiqui et al., J Allergy Clin Immunol, (2023).
98. Bakker et al., Crit Care Med, 32:1-12 (2004).
99. Miller et al., Int Immunol, 25:471-483 (2013).
100. Okazaki et al., Am J Transplant, 7:1672-1679 (2007).
101. Grimaldi et al., J Leukoc Biol, 65:846-853 (1999).
102. Grisaru-Tal et al., Cancer Res, 81:5555-5571 (2021).
103. Liu, et al., J Immunol, 174:5936-5940 (2005).
104. Ikutani et al., Immunobiology. 223(6-7):486-92 (2018).
105. Coffman et al. Science. 245(4915):308-10 (1989).
106. Jorssen et al. Immunity. 57(7):1549-66 e8, (2024).
107. Hellman et al. Clin Exp Immunol. 131(1):75-81, (2003)
108. Daley et al. J Leukoc Biol. 83(1):64-70, (2008).
109. Panopoulos et al. Cytokine. 42(3):277-88, (2008).
110. Zafra et al. Int J Mol Sci. 16(3):5434-51, (2015).
111. Yamaguchi et al. J Exp Med. 167(1):43-56, (1988).
112. Takatsu et al. Proc Jpn Acad Ser B Phys Biol Sci. 87(8):463-85, (2011).
113. Mei et al. Sci Transl Med. 16(761):eadl4222, (2024).
114. Rothenberg et al. Annu Rev Immunol. 24:147-74, (2006).
115. Pelaia et al. Front Physiol. 10:1514, (2019).
116. Kyriakopoulos et al. ERJ Open Res. 11(2), (2025).
117. Saqib et al. Cell Rep. 44(1):115072, (2025).
118. Coltoff et al. Blood Adv. 9(8):1907-15, (2025).
119. Bylund et al. Infect Immun. 70(6):2908-14, (2002.
120. Croker et al. Immunity. 20(2):153-65, (2004).
121. Lawrence et al. Microbiol Mol Biol Rev. 82(1) (2018).
122. Cowland et al. Immunol Rev. 273(1):11-28 (2016).
123. Pipkin et al. Immunol Rev. 235(1):55-72, (2010).
124. Chopp et al. J Allergy Clin Immunol. 151(1):81-97, (2023).

What is claimed:

1. A method for depleting an eosinophil population in a subject in need thereof, comprising:
administering at least one time to the subject an amount of an antibody directed against eosinophils in the population.

2. The method of claim 1, wherein the antibody directly targets and kills the eosinophil population.

3. The method of claim 1, wherein the antibody is a monoclonal antibody.

4. The method of claim 3, wherein the monoclonal antibody is an anti-IL-5Rα antagonistic antibody.

5. The method of claim 3, wherein the monoclonal antibody is selected from the group consisting of benralizumab, lirentelimab, and dexpramipexole.

6. The method of claim 1, wherein the subject in need underwent a medical procedure involving one or both lungs.

7. The method of claim 6, wherein the medical procedure is a lung resection, a lung transplantation, radiofrequency ablation of pulmonary tumors or radiotherapy of pulmonary tumors.

8. A method for inhibiting an onset of eosinophilia in a subject after a pulmonary medical procedure, comprising:
depleting eosinophils from a population thereof in the subject after the pulmonary medical procedure.

9. The method of claim 8, wherein the depleting step comprises:
targeting directly the eosinophils in the population with an antibody effective to reduce the number of the eosinophils therein.

10. The method of claim 8, wherein the antibody targets a cytokine associated with the eosinophil activation cascade.

11. The method of claim 8, wherein the antibody is an anti-IL-5Rα antagonistic antibody.

12. The method of claim 11, wherein the anti-IL-5Rα antagonistic antibody is selected from the group consisting of benralizumab, lirentelimab, and dexpramipexole.

13. The method of claim 8, wherein the pulmonary medical procedure is a lung resection.

14. An antibody-mediated method for preventing activation of lung-resident eosinophils in a subject after a lung resection surgery, comprising:
administering to the subject an amount of an anti-IL-5rα antagonistic antibody effective to directly target the lung-resident esoinophils.

15. The method of claim 14, wherein the anti-IL-5rα antagonistic antibody is a monoclonal antibody.

16. The method of claim 14, wherein the antibody is selected from the group consisting of benralizumab, lirentelimab, and dexpramipexole.

17. A post-surgical method for treating lung tissue after a procedure involving one or both lungs in a subject, comprising:
administering an amount of a monoclonal antibody to the subject effective to directly reduce the number of eosinophils in the lung tissue and concomitantly to prevent disinhibition of cytotoxicity of immature neutrophils therein.

18. The post-surgical method of claim 17, wherein the monoclonal antibody is an anti-IL-5rα antagonistic antibody that directly targets the eosinophils; wherein the antibody is not an anti-IL-5 antagonistic antibody.

19. The post-surgical method of claim 18, wherein the monoclonal antibody is benralizumab.

20. The post-surgical method of claim 17, wherein the procedure is a lung resection, a lung transplantation, radiofrequency ablation of pulmonary tumors or radiotherapy of pulmonary tumors.
